(19) United States
(12) Patent Application Publication
(10) Pub. No.: US 2024/0093250 A1
(43) Pub. Date: Mar. 21, 2024

(54) PROCESS FOR PREPARING A CONJUGATE LINKING MOIETY

(71) Applicant: Cybrexa 2, Inc., Haven, CT (US)

(72) Inventors: Robert John Maguire, New Haven, CT (US); Michiel Christian Alexander van Vliet, Den Hoorn (NL); Willem Robert Klaas Schoevaart, Den Hoorn (NL)

(21) Appl. No.: 18/271,121

(22) PCT Filed: Jan. 7, 2022
(86) PCT No.: PCT/US2022/011629

§ 371 Date: Jul. 6, 2023

Related U.S. Application Data
(60) Provisional application No. 63/135,088, filed on Jan. 8, 2021.

(51) Int. Cl. C12P 11/00; C12N 9/18; C12N 11/087

(52) U.S. Cl. CPC C12P 11/00; C12N 9/18; C12N 11/087; C12Y 301/01003

(57) ABSTRACT

The present invention relates to processes for preparing linkers that are useful in the conjugation of therapeutic molecules (e.g., cytotoxic agents) with targeting moieties (e.g., proteins, peptides, antibodies, nanoparticles, nucleic acids). During said processes lipases like lipase B from Candida antarctica were used for enantioselective resolution of (S,S)-2-benzylthiocyclohexanol or (S,S)-2-benzylthiocycloheptanol in presence of acylating agent which are reduced for deprotection to yield (S,S)-2-mercaptocyclohexanol or (S,S)-2-mercaptocyclopentanol which can then be used for linking therapeutic with targeting moieties.

### FIELD OF THE INVENTION

The present invention relates to processes for preparing linkers that are useful in the conjugation of therapeutic molecules (e.g., cytotoxic agents) with targeting moieties (e.g., proteins, peptides, antibodies, nanoparticles, nucleic acids).

### BACKGROUND OF THE INVENTION

Cancer is a group of diseases characterized by aberrant control of cell growth. The annual incidence of cancer is estimated to be in excess of 1.6 million in the United States alone. While surgery, radiation, chemotherapy, and hormones are used to treat cancer, it remains the second leading cause of death in the U.S., and additional strategies of treatment are needed. Drug conjugates have emerged as a viable and continuously explored approach to target malignant tumors.

Despite advances in the selectivity of chemotherapy drugs over the past several decades, traditional cytotoxic chemotherapy drugs often lack sufficient specificity and targeting effects, causing injury to normal, non-cancerous cells which can lead to serious adverse reactions. Drug conjugates, comprised of a drug (e.g., a cytotoxic agent) linked to a targeting moiety (e.g., a peptide, protein, or antibody) have been developed for use in tumor targeted therapy. Drug conjugates can provide for the preferential delivery of drug to diseased tissue, reducing undesired side effects such as damage to non-cancerous tissue. See, for example, Vrettos, V., “On the design principles of peptide-drug conjugates for targeted drug delivery to the malignant tumor site,” Beilstein J. Org. Chem. 2018, 14:930-954.

The development of linkers, groups which join the drug to the targeting moiety, has emerged as an important aspect in the design of new drug conjugates. Linkers are desirably stable enough in vivo to allow for delivery of the drug to the targeted diseased cell. In addition, the linker should not perturb the binding affinity of the targeting moiety to its target. Finally, after localization of the drug to the target, the linker should be able to release the drug so that the released drug may bind to its target. See Lu, J., “Linkers Having a Crucial Role in Antibody-Drug Conjugates,” Int. J. Mol. Sci. 2016, 17, 1-22; and Corso A. D., “Innovative Linker Strategies for Tumor-Targeted Drug Conjugates,” Chem. Eur. J. 2019, 25(65):14740-14757. Thus, there is a need for the development of new linkers and processes for making them.

### SUMMARY

Provided herein is a process for preparing a compound of Formula (A1)

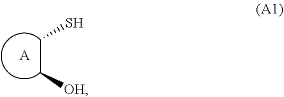

or a salt thereof, wherein ring A is C₅₋₇ cycloalkyl or 5-7 membered heterocycloalkyl, comprising:
a) treating a compound of Formula (A4)

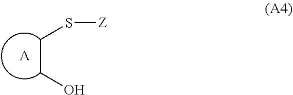

or a salt thereof, wherein Z is a protecting group, with Ak1, wherein Ak1 is an acylating reagent, in the presence of an enzyme to provide a mixture of a compound of Formula (A2) and a compound of Formula (A3);

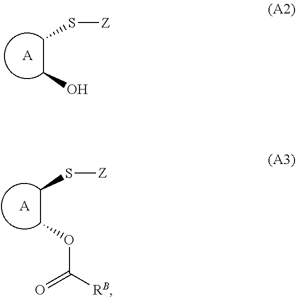

or salts thereof wherein R^{B} is C₁₋₆ alkyl optionally substituted with COOH; and
b) deprotecting the compound of Formula (A2), or a salt thereof, to provide a compound of Formula (A1), or a salt thereof.

Also provided herein is a process for preparing a compound of Formula (A-I):

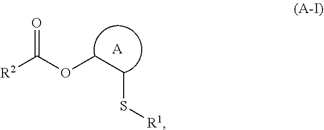

or a pharmaceutically acceptable salt thereof, wherein
ring A is C₅₋₇ cycloalkyl or 5-7 membered heterocycloalkyl;
R¹ is a targeting moiety; and
R² is a therapeutic moiety;
   comprising:
a) reacting a compound of Formula (A1), or a salt thereof, prepared by the process of any one of claims 1-48, with R^{C}—S—S—R^{C} to provide a compound of Formula (A8)

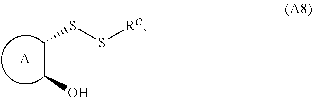

or a salt thereof, wherein R^{C} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃;
b) reacting a compound of Formula (A8), or a salt thereof, with R^{E}OC(O)OR^{E}, wherein R^{E} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃; to provide a compound of Formula (A-1B)

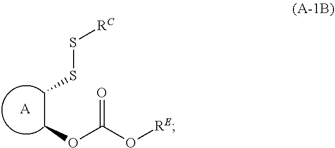

or a salt thereof;
c) reacting the compound of formula (A-1B) or a salt thereof, with R²H to provide a compound of Formula (A-1C)

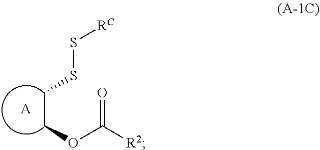

or a salt thereof; and
reacting a compound of Formula (A-1C), or a salt thereof, with R¹H to provide a compound of Formula (A-I).

### DETAILED DESCRIPTION

### Preparation of Compounds of Formula (A1)

Provided herein is a process for preparing a compound of Formula (A1)

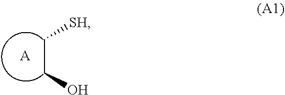

or a salt thereof, wherein ring A is C₅₋₇ cycloalkyl or 5-7 membered heterocycloalkyl, which is useful for preparing conjugates of therapeutic agents such as, e.g., cytotoxic agents.

Processes for preparing compounds of Formula (A1) are described in U.S. Patent Publication No. US 2019/209580, U.S. patent application Ser. No. 16/925,094 (U.S. Publication No. 2021/0009719), and U.S. patent application Ser. No. 16/924,445 (U.S. Publication No. 2021/0009536). The present disclosure provides advantages over previously disclosed processes for preparing compounds of Formula (A1), offering better yields, scalability, and requiring less intensive procedures for purification. In particular, the present disclosure provides for the enzyme-catalyzed resolution of the enantiomers of compounds of Formula (A1). The disclosure further provides the enantioselective synthesis of compounds of Formula (A4), which are precursors to the compounds of Formula (A1). In contrast, prior disclosed processes relied on the separation of enantiomers via HPLC or with chiral chromatography, which is more difficult to perform on a large scale and more costly than the processes provided herein.

Provided herein is a process for preparing a compound of Formula (A1)

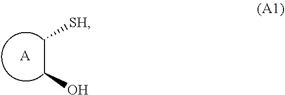

or a salt thereof, wherein ring A is C₅₋₇ cycloalkyl or 5-7 membered heterocycloalkyl, comprising:
a) treating a compound of Formula (A4)

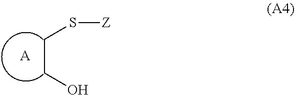

or a salt thereof, wherein Z is a protecting group, with Ak1, wherein Ak1 is an acylating reagent, in the presence of an enzyme to provide a mixture of a compound of Formula (A2) and a compound of Formula (A3);

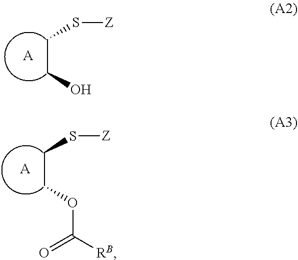

or salts thereof wherein R^{B} is C₁₋₆ alkyl optionally substituted with COOH; and
b) deprotecting the compound of Formula (A2), or a salt thereof, to provide a compound of Formula (A1), or a salt thereof.

In some embodiments, Ring A is C₅₋₇ cycloalkyl. In some embodiments, Ring A is cyclopentyl. In some embodiments, Ring A is cyclohexyl. In some embodiments, Ring A is cycloheptyl.

In some embodiments, Ring A is C₅₋₇ cycloalkyl. In some embodiments, Ring A is cyclopentyl. In some embodiments, Ring A is cyclohexyl. In some embodiments, Ring A is cycloheptyl.

In some embodiments, Ring A is 5-7 membered heterocycloalkyl. In some embodiments, Ring A is 5-membered heterocycloalkyl. In some embodiments, Ring A is 6-membered heterocycloalkyl. In some embodiments, Ring A is 7-membered heterocycloalkyl. In some embodiments, Ring A is tetrahydrofuranyl. In some embodiments, Ring A is tetrahydropyranyl.

Also provided herein is a process for preparing a compound of Formula (1)

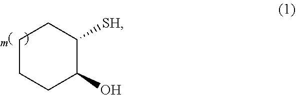

or a salt thereof, wherein m is 0, 1, or 2, comprising:
a) treating a compound of Formula (4)

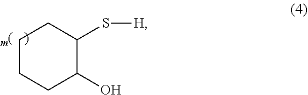

or a salt thereof, wherein Z is a protecting group,
with Ak1, wherein Ak1 is an acylating reagent, in the presence of an enzyme to provide a mixture of a compound of Formula (2) and a compound of Formula (3);

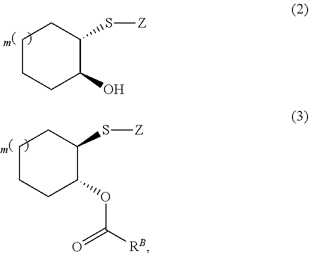

or salts thereof wherein R^{B} is C₁₋₆ alkyl optionally substituted with COOH; and
b) deprotecting the compound of Formula (2), or a salt thereof, to provide a compound of Formula (1), or a salt thereof.

In some embodiments, m is 0. In some embodiments, m is 1. In some embodiments, m is 2.

In some embodiments, Z is —CH₂R^{A}, wherein R^{A} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents independently selected from C₁₋₄ alkyl, halo, CN, NO₂, OH, and OCH₃.

In some embodiments, R^{A} is C₆₋₁₀ aryl. In some embodiments, R^{A} is phenyl.

In some embodiments, Ak1 is glutaric anhydride, succinic anhydride, or isopropenyl acetate. In some embodiments, Ak1 is glutaric anhydride. In some embodiments, Ak1 is succinic anhydride. In some embodiments, Ak1 is isopropenyl acetate.

In some embodiments, R^{B} is CH₃, CH₂CH₂COOH, or CH₂CH₂CH₂COOH. In some embodiments, R^{B} is CH₃. In some embodiments, R^{B} is CH₂CH₂COOH. In some embodiments, R^{B} is CH₂CH₂CH₂COOH.

As used herein, the term “enzyme” refers to a protein that catalyzes chemical reactions. In some embodiments, the enzyme can catalyze esterification (e.g., the formation of an ester from an alcohol) reactions. In some embodiments, the enzyme can catalyze esterification reactions in an enantioselective manner (e.g., favoring the formation of one enantiomer over the opposing enantiomer). In some embodiments, the enzyme is a lipase enzyme.

As used herein, the term “lipase enzyme” refers to an enzyme that in natural conditions (e.g., in aqueous media) catalyzes the hydrolysis of lipids. In nonaqueous media (e.g., organic solvents), certain lipase enzymes can catalyze esterification reactions (e.g., the conversion of alcohols into esters). Lipase enzymes that are capable of catalyzing esterification reactions in organic solvents in an enantioselective manner (e.g., favoring the formation of one enantiomer over the opposing enantiomer) are known in the art. See, for example, Kumar et al., “Lipase catalysis in organic solvents: advantages and applications,” Biol. Proced. Online 2016; 18:2; Ducret, A., “Lipase-catalyzed enantioselective esterification of ibuprofen in organic solvents under controlled water activity,” Enzyme and Microbial Technology 1998, 22(4):212-216.

In some embodiments, the enzyme is immobilized on a solid support (i.e., bound to a solid that is insoluble in the reaction media). The enzyme can be bound to the solid support through, e.g., covalent binding to functional groups on the solid support, adsorption onto the solid support, and entrapment or encapsulation on the solid support. Immobilization on a solid substrate can increase enzyme stability and facilitate the recovery of products and recycling of enzymes. In some embodiments, the solid support is silica or an inorganic oxide. In some embodiments, the solid support is an activated carbon or a modified or unmodified charcoal. In some embodiments, the solid support is a synthetic polymer (e.g., amino and carboxyl-plasma activated polypropylene film; and copolymers of methacrylate). In some embodiments, the solid support is Immobead. In some embodiments, the solid support is an ion exchange resin (e.g., Amberlite and Sepabeads). In some embodiments, the solid support is silica gel. In some embodiments, the solid support is polystyrene. In some embodiments, the solid support is cellulose nanocrystals. In some embodiments, the solid support is chitosan. In some embodiments, the solid support is an acrylic bead. Enzyme immobilization techniques are well known in the art. See Zdarta, J., “A General Overview of Support Materials for Enzyme Immobilization: Characteristics, Properties, Practical Utility,” Catalysts 2018, 8, 92, 1-27; and Miletic, N., “Immobilization of Candida antarctica lipase B on polystyrene nanoparticles,” Macromolecular Rapid Communications, 2010, 31(1):71-74.

In some embodiments, the enzyme is a lipase enzyme derived from a bacterial or fungal source. In some embodiments, the enzyme is a lipase enzyme derived from a fungal source. In some embodiments, the enzyme is a lipase enzyme derived from a bacterial source. In some embodiments, the enzyme is a lipase enzyme derived from Candida antarctica, Rhizomucor miehei, Thermomyces lanuginosa, Candida rugosa, Pseudomonas cepacia, Pseudomonas fluorescens, Rhizopus oryzae, Mucor javanicus, Aspergillus niger, Rhizopus niveus, Alcaligenes sp., Resinase HT, Lipex 100L, Novozymes Stickaway, Candida cylindracea sp., or Bacillus subtilis. In some embodiments, the enzyme is a lipase enzyme derived from Candida antarctica. In some embodiments, the enzyme is Candida antarctica lipase B. Enzymes can be obtained from Novozymes, Genencor, Sigma-Aldrich, c-Lecta, Aum Enzymes and immobilized on a solid substrate such as, for example, Immobead COV-1.

In some embodiments, the enzyme is selected from one of the following:
lipase A from Candida antarctica covalently attached to dry acrylic beads;
lipase B from Candida antarctica covalently attached to dry acrylic beads;
generic lipase B from Candida antarctica covalently attached to dry acrylic beads;
lipase from Rhizomucor miehei covalently attached to dry acrylic beads;
lipase from Thermomyces lanuginosa covalently attached to dry acrylic beads;
lipase from Candida rugosa covalently attached to dry acrylic beads;
lipase from Pseudomonas cepacia covalently attached to dry acrylic beads;
lipase from Pseudomonas fluorescens covalently attached to dry acrylic beads;
lipase from Rhizopus oryzae covalently attached to dry acrylic beads;
lipase from Mucor javanicus covalently attached to dry acrylic beads;
lipase from Aspergillus niger covalently attached to dry acrylic beads;
lipase from Rhizopus niveus covalently attached to dry acrylic beads;
lipase from Alcaligenes sp. covalently attached to dry acrylic beads;
lipase Resinase HT covalently attached to dry acrylic beads;
lipase Lipex 100L covalently attached to dry acrylic beads;
lipase from Fusarium solani pisi, Novozyme 51032 covalently attached to dry acrylic beads;
lipase from Candida cylindracea sp. covalently attached to dry acrylic beads; and
lipase from Bacillus subtilis covalently attached to dry acrylic beads.

In some embodiments, the enzyme is selected from one of the following:
ChiralVision Product No. IMMCALA-T2-150;
ChiralVision Product No. IMMCALB-T2-150;
ChiralVision Product No. IMMCALBY-T2-150;
ChiralVision Product No. IMMRML-T2-150;
ChiralVision Product No. IMMTLL-T2-150;
ChiralVision Product No. IMMCRL-T2-150;
ChiralVision Product No. IMMABC-T2-150;
ChiralVision Product No. IMMAPF-T2-150;
ChiralVision Product No. IMMARO-T2-150;
ChiralVision Product No. IMMAMJ-T2-150;
ChiralVision Product No. IMMANA-T2-150;
ChiralVision Product No. IMMRNA-T2-150;
ChiralVision Product No. IMMASMQ-T2-150;
ChiralVision Product No. IMMRES-T2-150;
ChiralVision Product No. IMMLIPX-T2-150;
ChiralVision Product No. IMML51-T2-150;
ChiralVision Product No. IMMCCMO-T2-150;
ChiralVision Product No. IMMAULI-T2-150; and
ChiralVision Product No. CaLB-ADS4,

In some embodiments, the enzyme is ChiralVision Product No. IMMCALB-T2-150. In some embodiments, the enzyme is ChiralVision Product No. CaLB-ADS4.

The treating of a compound of Formula (A4) with Ak1 can be performed at a temperature between about 15° C. and about 20° C. In some embodiments, the treating of a compound of Formula (A4) with Ak1 is performed at room temperature.

The treating of a compound of Formula (A4) with Ak1 can be performed for a period of about 6 h to about 24 h. In some embodiments, the treating of a compound of Formula (A4) with Ak1 is performed for a period of about 16 h.

The treating of a compound of Formula (A4) with Ak1 can be performed in the presence of S1, wherein S1 is a solvent. In some embodiments, S1 is an ether solvent. In some embodiments, S1 is methyl tert-butyl ether. In some embodiments, S1 is 2-methyltetrahydrofuran.

The process can further comprise the step of separating the compound of Formula (A2) from the compound of Formula (A3). In some embodiments, the separating comprises treating the mixture with an aqueous base and separating the aqueous layer from the mixture. In some embodiments, the aqueous base is aqueous sodium carbonate.

In some embodiments, Z is —CH₂R^{A}, and the deprotecting comprises reducing the compound of Formula (A2) with RA1, wherein RA1 is a reducing agent. In some embodiments, R^{A} is phenyl.

In some embodiments, RA1 is lithium metal, sodium metal, or calcium metal. In some embodiments, RA1 is lithium metal.

The reducing can be carried out in the presence of S2, wherein S2 is a solvent. In some embodiments, S2 is an ether solvent. In some embodiments, S2 is 2-methyltetrahydrofuran.

In some embodiments, the compound of Formula (A1) is isolated in greater than 75% enantiomeric excess. In some embodiments, the compound of Formula (A1) is isolated in greater than 90% enantiomeric excess. In some embodiments, the compound of Formula (A1) is isolated in greater than 95% enantiomeric excess. In some embodiments, the compound of Formula (A1) is isolated in greater than 99% enantiomeric excess.

In some embodiments, Compound 1 is isolated in greater than 75% enantiomeric excess. In some embodiments, Compound 1 is isolated in greater than 90% enantiomeric excess. In some embodiments, Compound 1 is isolated in greater than 95% enantiomeric excess. In some embodiments, Compound 1 is isolated in greater than 99% enantiomeric excess.

The compound of Formula (A4) can be prepared by a process comprising reacting a compound of Formula (A5)

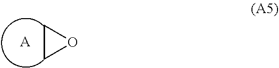

or a salt thereof, with R^{A}CH₂SH (Formula (6)), or a salt thereof, to provide the compound of Formula (A4) or a salt thereof, wherein R^{A} is as defined herein.

In some embodiments, the reacting of the compound of Formula (A5) or a salt thereof with R^{A}CH₂SH (Formula (6)), or a salt thereof is performed in the presence of M1, wherein M1 is a metal catalyst. In some embodiments, M1 is a zinc salt. In some embodiments, M1 is zinc (D)-tartrate.

The reacting of the compound of Formula (A5) or a salt thereof with R^{A}CH₂SH (Formula (6)) can be performed in the presence of B1, wherein B1 is a base. In some embodiments, B1 is an alkoxide base. In some embodiments, B1 is sodium ethoxide.

The reacting of the compound of Formula (A5) with a compound of Formula (A6) can be performed in the presence of S3, wherein S3 is a solvent. In some embodiments, S3 is a halogenated solvent or an ether solvent. In some embodiments, S3 is dichloromethane. In some embodiments, S3 is 2-methyltetrahydrofuran.

In some embodiments, the compound of Formula (A4) is isolated in greater than 25% enantiomeric excess. In some embodiments, the compound of Formula (A4) is isolated in greater than 50% enantiomeric excess. In some embodiments, the compound of Formula (A4) is isolated in greater than 70% enantiomeric excess. In some embodiments, the compound of Formula (A4) is isolated in greater than 80% enantiomeric excess. In some embodiments, the compound of Formula (A4) is isolated in greater than 90% enantiomeric excess. In some embodiments, the compound of Formula (A4) is isolated in greater than 95% enantiomeric excess. In some embodiments, the compound of Formula (A4) is isolated in greater than 99% enantiomeric excess.

The compound of Formula (4) can be prepared by a process comprising reacting a compound of Formula (5)

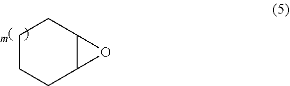

or a salt thereof, with R^{A}CH₂SH (Formula (6)), or a salt thereof, to provide the compound of Formula (4) or a salt thereof, wherein m and R^{A} are as defined herein.

In some embodiments, m is 0. In some embodiments, m is 1. In some embodiments, m is 2.

In some embodiments, the reacting of the compound of Formula (5) or a salt thereof with R^{A}CH₂SH (Formula (6)), or a salt thereof is performed in the presence of M1, wherein M1 is a metal catalyst. In some embodiments, M1 is a zinc salt. In some embodiments, M1 is zinc (D)-tartrate.

The reacting of the compound of Formula (5) or a salt thereof with R^{A}CH₂SH (Formula (6)) can be performed in the presence of B1, wherein B1 is a base. In some embodiments, B1 is an alkoxide base. In some embodiments, B1 is sodium ethoxide.

The reacting of the compound of Formula (5) with a compound of Formula (6) can be performed in the presence of S3, wherein S3 is a solvent. In some embodiments, S3 is a halogenated solvent or an ether solvent. In some embodiments, S3 is dichloromethane. In some embodiments, S3 is 2-methyltetrahydrofuran.

In some embodiments, the compound of Formula (4) is isolated in greater than 25% enantiomeric excess. In some embodiments, the compound of Formula (4) is isolated in greater than 50% enantiomeric excess. In some embodiments, the compound of Formula (4) is isolated in greater than 70% enantiomeric excess. In some embodiments, the compound of Formula (4) is isolated in greater than 80% enantiomeric excess. In some embodiments, the compound of Formula (4) is isolated in greater than 90% enantiomeric excess. In some embodiments, the compound of Formula (4) is isolated in greater than 95% enantiomeric excess. In some embodiments, the compound of Formula (4) is isolated in greater than 99% enantiomeric excess.

In some embodiments, the Compound of Formula (A1) is a compound of Formula (1)

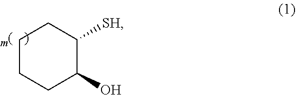

or a salt thereof, wherein m is 0, 1, or 2.

In some embodiments, the Compound of Formula (A2) is a compound of Formula (2)

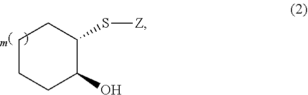

or a salt thereof, wherein m is 0, 1, or 2.

In some embodiments, the Compound of Formula (A3) is a compound of Formula (3)

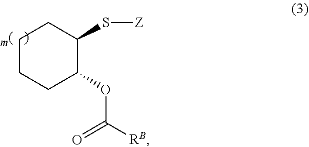

or a salt thereof, wherein m is 0, 1, or 2.

In some embodiments, the Compound of Formula (A4) is a compound of Formula (4)

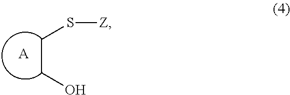

or a salt thereof, wherein m is 0, 1, or 2.

In some embodiments, the Compound of Formula (A5) is a compound of Formula (5)

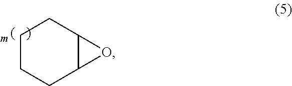

or a salt thereof, wherein m is 0, 1, or 2.

In some embodiments, m is 0. In some embodiments, m is 1. In some embodiments, m is 2.

In some embodiments, the compound of Formula (1) is Compound 1:

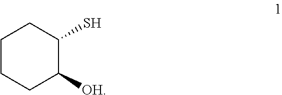

In some embodiments, the compound of Formula (2) is Compound 2:

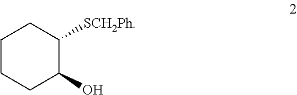

In some embodiments, the compound of Formula (3) is Compound 3:

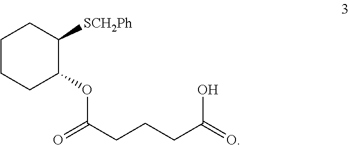

In some embodiments, the compound of Formula (4) is Compound 4:

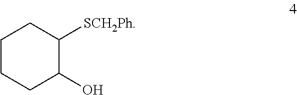

In some embodiments, the compound of Formula (5) is Compound 5:

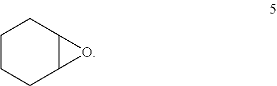

In some embodiments, the compound of Formula (6) is benzyl mercaptan.

Also provided herein is a process for preparing Compound 1 having the formula:

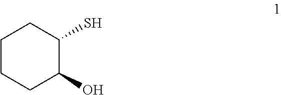

or a salt thereof, comprising:
a) reacting Compound 5 having the formula:

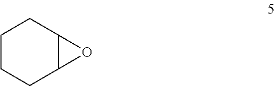

or a salt thereof, with benzyl mercaptan to provide Compound 4 having the formula:

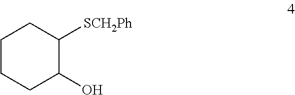

or a salt thereof;
b) treating Compound 4 with glutaric anhydride in the presence of an enzyme to provide a mixture of Compound 2 and Compound 3;

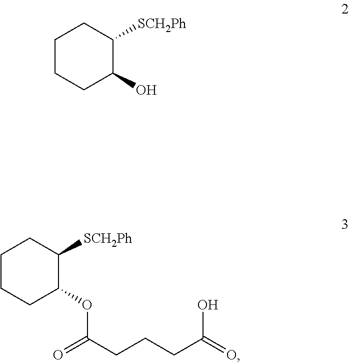

or salts thereof; and
c) reducing Compound 2 to provide Compound 1.

In some embodiments, the enzyme is a lipase enzyme.

Also provided herein is a compound of Formula (A1) prepared by any of the processes for preparing a compound of Formula (A1) described herein.

### Preparation of Compound A8 and Compound 8

Provided herein is a process for preparing a compound of Formula A8, which is a conjugate linker that is useful in preparing conjugates as therapeutics.

Provided herein is a process for preparing a compound of Formula (A8):

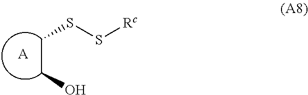

or a salt thereof, wherein:
ring A is C₅₋₇ cycloalkyl or 5-7 membered heterocycloalkyl;
R^{C} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents independently selected from C₁₋₄ alkyl, halo, CN, NO₂, OH, and OCH₃,
comprising reacting a compound of Formula (A7)

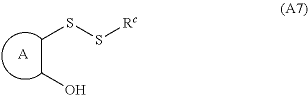

or a salt thereof, with Ak2, wherein Ak2 is an acylating reagent, in the presence of an enzyme to provide a mixture of a compound of Formula (A8) and a compound of Formula (A9);

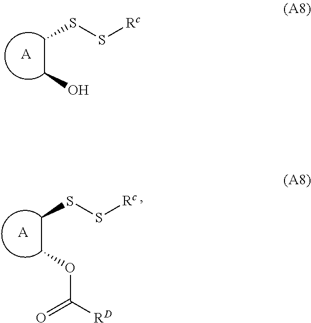

or salts thereof, wherein R^{D} is C₁₋₆ alkyl optionally substituted with COOH.

Provided herein is a process for preparing Compound 8, which is a conjugate linker that is useful in preparing conjugates as therapeutics.

Provided herein is a process for preparing a compound of Formula (8):

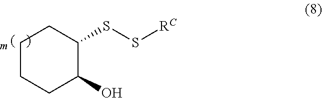

or a salt thereof, wherein m is 0, 1, or 2, and R^{C} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents independently selected from C₁₋₄ alkyl, halo, CN, NO₂, OH, and OCH₃, comprising reacting a compound of Formula (7)

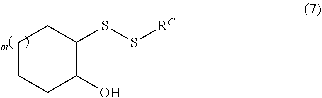

or a salt thereof, with Ak2, wherein Ak2 is an acylating reagent, in the presence of an enzyme to provide a mixture of a compound of Formula (8) and a compound of Formula (9);

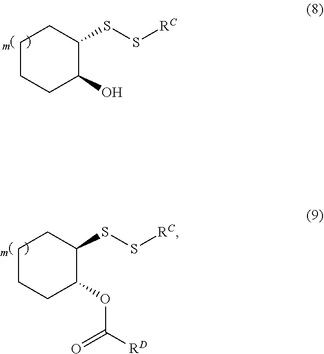

or salts thereof, wherein R^{D} is C₁₋₆ alkyl optionally substituted with COOH.

Compounds of Formula (A7) and Formula (7), and processes for preparing thereof, are described in U.S. Patent Publication No. US 2019/209580, U.S. patent application Ser. No. 16/925,094, and U.S. patent application Ser. No. 16/924,445.

In some embodiments, m is 0. In some embodiments, m is 1. In some embodiments, m is 2.

In some embodiments, Ak2 is glutaric anhydride, succinic anhydride, or isopropenyl acetate. In some embodiments, Ak2 is glutaric anhydride. In some embodiments, Ak2 is succinic anhydride. In some embodiments, Ak2 is isopropenyl acetate.

In some embodiments, R^{C} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃. In some embodiments, R^{C} is 5-10 membered heteroaryl optionally substituted with 1, 2, 3, 4, or 5 substituents selected from C₁₋₄ alkyl, halo, CN, NO₂, OH, and OCH₃. In some embodiments, R^{C} is 5-10 membered heteroaryl. In some embodiments, R^{C} is pyridinyl.

In some embodiments, R^{D} is CH₃, CH₂CH₂COOH, or CH₂CH₂CH₂COOH. In some embodiments, R^{D} is CH₃. In some embodiments, R^{D} is CH₂CH₂COOH. In some embodiments, R^{D} is CH₂CH₂CH₂COOH.

In some embodiments, the enzyme is a lipase enzyme.

In some embodiments, the enzyme is immobilized on a solid support (i.e., bound to a solid that is insoluble in the reaction media). The enzyme can be bound to the solid support through, e.g., covalent binding to functional groups on the solid support, adsorption onto the solid support, and entrapment or encapsulation on the solid support. Immobilization on a solid substrate can increase enzyme stability and facilitate the recovery of products and recycling of enzymes. In some embodiments, the solid support is silica or an inorganic oxide. In some embodiments, the solid support is an activated carbon or a modified or unmodified charcoal. In some embodiments, the solid support is a synthetic polymer (e.g., amino and carboxyl-plasma activated polypropylene film; and copolymers of methacrylate). In some embodiments, the solid support is Immobead. In some embodiments, the solid support is an ion exchange resin (e.g., Amberlite and Sepabeads). In some embodiments, the solid support is silica gel. In some embodiments, the solid support is polystyrene. In some embodiments, the solid support is cellulose nanocrystals. In some embodiments, the solid support is chitosan. In some embodiments, the solid support is an acrylic bead. Enzyme immobilization techniques are well known in the art. See Zdarta, J., “A General Overview of Support Materials for Enzyme Immobilization: Characteristics, Properties, Practical Utility,” Catalysts 2018, 8, 92, 1-27; and Miletic, N., “Immobilization of Candida antarctica lipase B on polystyrene nanoparticles,” Macromolecular Rapid Communications, 2010, 31(1):71-74.

In some embodiments, the enzyme is a lipase enzyme derived from a bacterial or fungal source. In some embodiments, the enzyme is a lipase enzyme derived from a fungal source. In some embodiments, the enzyme is a lipase enzyme derived from a bacterial source. In some embodiments, the enzyme is a lipase enzyme derived from Candida antarctica, Rhizomucor miehei, Thermomyces lanuginosa, Candida rugosa, Pseudomonas cepacia, Pseudomonas fluorescens, Rhizopus oryzae, Mucor javanicus, Aspergillus niger, Rhizopus niveus, Alcaligenes sp., Resinase HT, Lipex 100L, Novozymes Stickaway, Candida cylindracea sp., or Bacillus subtilis. In some embodiments, the enzyme is a lipase enzyme derived from Candida antarctica. In some embodiments, the enzyme is Candida antarctica lipase B. Enzymes can be obtained from Novozymes, Genencor, Sigma-Aldrich, c-Lecta, Aum Enzymes and immobilized on a solid substrate such as, for example, Immobead COV-1.

In some embodiments, the enzyme is selected from one of the following:
lipase A from Candida antarctica covalently attached to dry acrylic beads;
lipase B from Candida antarctica covalently attached to dry acrylic beads;
generic lipase B from Candida antarctica covalently attached to dry acrylic beads;
lipase from Rhizomucor miehei covalently attached to dry acrylic beads;
lipase from Thermomyces lanuginosa covalently attached to dry acrylic beads;
lipase from Candida rugosa covalently attached to dry acrylic beads;
lipase from Pseudomonas cepacia covalently attached to dry acrylic beads;
lipase from Pseudomonas fluorescens covalently attached to dry acrylic beads;
lipase from Rhizopus oryzae covalently attached to dry acrylic beads;
lipase from Mucor javanicus covalently attached to dry acrylic beads;
lipase from Aspergillus niger covalently attached to dry acrylic beads;
lipase from Rhizopus niveus covalently attached to dry acrylic beads;
lipase from Alcaligenes sp. covalently attached to dry acrylic beads;
lipase Resinase HT covalently attached to dry acrylic beads;
lipase Lipex 100L covalently attached to dry acrylic beads;
lipase from Fusarium solani pisi, Novozyme 51032 covalently attached to dry acrylic beads;
lipase from Candida cylindracea sp. covalently attached to dry acrylic beads; and
lipase from Bacillus subtilis covalently attached to dry acrylic beads.

In some embodiments, the enzyme is selected from one of the following:
ChiralVision Product No. IMMCALA-T2-150;
ChiralVision Product No. IMMCALB-T2-150;
ChiralVision Product No. IMMCALBY-T2-150;
ChiralVision Product No. IMMRML-T2-150;
ChiralVision Product No. IMMTLL-T2-150;
ChiralVision Product No. IMMCRL-T2-150;
ChiralVision Product No. IMMABC-T2-150;
ChiralVision Product No. IMMAPF-T2-150;
ChiralVision Product No. IMMARO-T2-150;
ChiralVision Product No. IMMAMJ-T2-150;
ChiralVision Product No. IMMANA-T2-150;
ChiralVision Product No. IMMRNA-T2-150;
ChiralVision Product No. IMMASMQ-T2-150;
ChiralVision Product No. IMMRES-T2-150;
ChiralVision Product No. IMMLIPX-T2-150;
ChiralVision Product No. IMML51-T2-150;
ChiralVision Product No. IMMCCMO-T2-150;
ChiralVision Product No. IMMAULI-T2-150; and
ChiralVision Product No. CaLB-ADS4,

In some embodiments, the enzyme is ChiralVision Product No. IMMCALB-T2-150. In some embodiments, the enzyme is ChiralVision Product No. CaLB-ADS4.

The treating of a compound of Formula (A7) with Ak2 can be performed at a temperature between about 15° C. and about 20° C. In some embodiments, the treating of a compound of Formula (A7) with Ak2 is performed at room temperature.

The treating of a compound of Formula (A7) with Ak2 can be performed for a period of about 12 h to about 60 h. In some embodiments, the treating of a compound of Formula (A7) with Ak2 is performed for a period of about 24 h to about 48 h. In some embodiments, the treating of a compound of Formula (A7) with Ak2 is performed for a period of about 48 h. In some embodiments, the treating of a compound of Formula (A7) with Ak2 is performed for a period of about 42 h.

The treating of a compound of Formula (A7) with Ak1 can be performed in the presence of S4, wherein S4 is a solvent. In some embodiments, S4 is an ether solvent. In some embodiments, S4 is methyl tert-butyl ether. In some embodiments, S4 is 2-methyltetrahydrofuran.

The process can further comprise the step of separating the compound of Formula (A8) from the compound of Formula (A9). In some embodiments, the separating comprises treating the mixture with an aqueous base and separating the aqueous layer from the mixture. In some embodiments, the aqueous base is aqueous sodium carbonate.

In some embodiments, the compound of Formula (A8) is isolated in greater than 25% enantiomeric excess. In some embodiments, the compound of Formula (A8) is isolated in greater than 50% enantiomeric excess. In some embodiments, the compound of Formula (A8) is isolated in greater than 70% enantiomeric excess. In some embodiments, the compound of Formula (A8) is isolated in greater than 80% enantiomeric excess. In some embodiments, the compound of Formula (A8) is isolated in greater than 90% enantiomeric excess. In some embodiments, the compound of Formula (A8) is isolated in greater than 95% enantiomeric excess. In some embodiments, the compound of Formula (A8) is isolated in greater than 99% enantiomeric excess.

The treating of a compound of Formula (7) with Ak2 can be performed at a temperature between about 15° C. and about 20° C. In some embodiments, the treating of a compound of Formula (7) with Ak2 is performed at room temperature.

The treating of a compound of Formula (7) with Ak2 can be performed for a period of about 12 h to about 60 h. In some embodiments, the treating of a compound of Formula (7) with Ak2 is performed for a period of about 24 h to about 48 h. In some embodiments, the treating of a compound of Formula (7) with Ak2 is performed for a period of about 48 h. In some embodiments, the treating of a compound of Formula (7) with Ak2 is performed for a period of about 42 h.

The treating of a compound of Formula (7) with Ak1 can be performed in the presence of S4, wherein S4 is a solvent. In some embodiments, S4 is an ether solvent. In some embodiments, S4 is methyl tert-butyl ether. In some embodiments, S4 is 2-methyltetrahydrofuran.

The process can further comprise the step of separating the compound of Formula (8) from the compound of Formula (9). In some embodiments, the separating comprises treating the mixture with an aqueous base and separating the aqueous layer from the mixture. In some embodiments, the aqueous base is aqueous sodium carbonate.

In some embodiments, the compound of Formula (8) is isolated in greater than 25% enantiomeric excess. In some embodiments, the compound of Formula (8) is isolated in greater than 50% enantiomeric excess. In some embodiments, the compound of Formula (8) is isolated in greater than 70% enantiomeric excess. In some embodiments, the compound of Formula (8) is isolated in greater than 80% enantiomeric excess. In some embodiments, the compound of Formula (8) is isolated in greater than 90% enantiomeric excess. In some embodiments, the compound of Formula (8) is isolated in greater than 95% enantiomeric excess. In some embodiments, the compound of Formula (8) is isolated in greater than 99% enantiomeric excess.

In some embodiments, the compound of Formula (A7) is Compound 7:

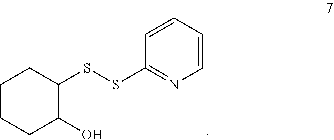 [SMILES: OC1CCCCC1SSc1ccccn1]

In some embodiments, the compound of Formula (A8) is Compound 8:

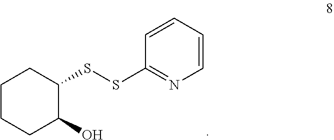 [SMILES: O[C@H]1CCCC[C@@H]1SSc1ccccn1]

In some embodiments, the compound of Formula (A9) is Compound 9:

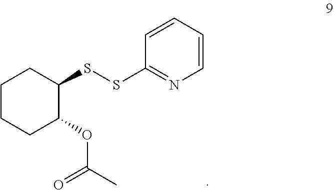 [SMILES: CC(=O)O[C@@H]1CCCC[C@H]1SSc1ccccn1]

In another embodiment, provided herein is a process for preparing Compound 8 having the formula:

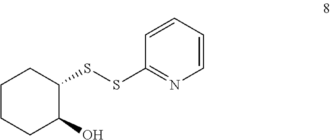 [SMILES: O[C@H]1CCCC[C@@H]1SSc1ccccn1]

or a salt thereof, comprising reacting Compound 7 having the formula:

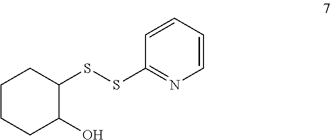 [SMILES: OC1CCCCC1SSc1ccccn1]

or a salt thereof, with isopropenyl acetate in the presence of an enzyme to provide a mixture of Compound 8 and Compound 9;

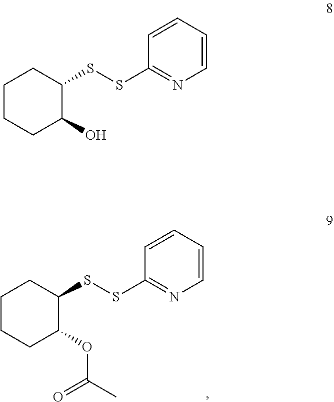 [SMILES: O[C@H]1CCCC[C@@H]1SSc1ccccn1 ; CC(=O)O[C@@H]1CCCC[C@H]1SSc1ccccn1]

or salts thereof.

In some embodiments, Compound 8 is isolated in greater than 25% enantiomeric excess. In some embodiments, Compound 8 is isolated in greater than 50% enantiomeric excess. In some embodiments, Compound 8 is isolated in greater than 70% enantiomeric excess. In some embodiments, Compound 8 is isolated in greater than 80% enantiomeric excess. In some embodiments, Compound 8 is isolated in greater than 90% enantiomeric excess. In some embodiments, Compound 8 is isolated in greater than 95% enantiomeric excess. In some embodiments, Compound 8 is isolated in greater than 99% enantiomeric excess.

Also provided herein is a compound of Formula (A8) prepared by any of the processes for preparing a compound of Formula (A8) described herein.

### Preparation of a Compound of Formula (I)

Provided herein is a process for preparing a compound of Formula (A-I):

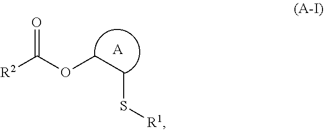

or a pharmaceutically acceptable salt thereof, wherein
ring A is a C₅₋₇ cycloalkyl group or 5-7 membered heterocycloalkyl group;
R¹ is a targeting moiety; and
R² is a therapeutic moiety;
   comprising:
a) reacting a compound of Formula (A1), or a salt thereof, which is prepared by the process disclosed herein, with R^{C}—S—S—R^{C} to provide a compound of Formula (A8)

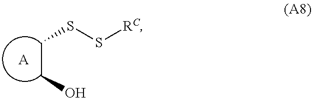

or a salt thereof, wherein R^{C} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃;
b) reacting a compounds of Formula (A8), or a salt thereof, with R^{E}OC(O)R^{F}, to provide a compound of Formula (A-1B)

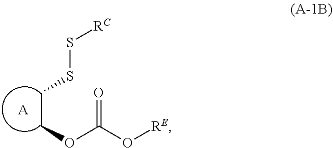

or a salt thereof, wherein:
R^{E} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃; and
R^{F} is halo or OR^{F1}, wherein OR^{F1} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃;
c) reacting the compound of formula (A-1B) or a salt thereof, with R²H to provide a compound of Formula (A-1C)

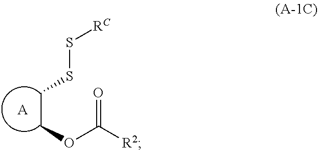

or a salt thereof; and
d) reacting a compound of Formula (A-1C), or a salt thereof, with R¹H to provide a compound of Formula (A-I).

Provided herein is a process for preparing a compound of Formula (I):

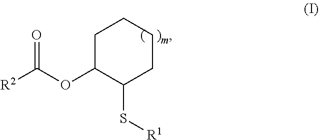

or a pharmaceutically acceptable salt thereof, wherein
R¹ is a targeting moiety;
R² is a therapeutic moiety; and
m is 0, 1, or 2;
   comprising:
a) reacting a compound of Formula (1), or a salt thereof, which is prepared by the process disclosed herein, with R^{C}—S—S—R^{C} to provide a compound of Formula (8)

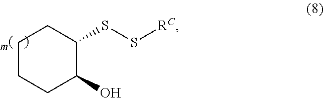

or a salt thereof, wherein R^{C} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃;
b) reacting a compound of Formula (8), or a salt thereof, with R^{E}OC(O)R^{F}, to provide a compound of Formula (1B)

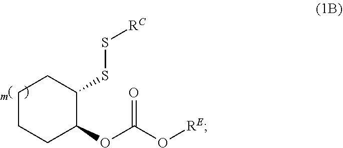

or a salt thereof, wherein:
R^{E} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃; and
R^{F} is halo or OR^{F1}, wherein OR^{F1} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃;
c) reacting the compound of formula (1B) or a salt thereof, with R²H to provide a compound of Formula (1C)

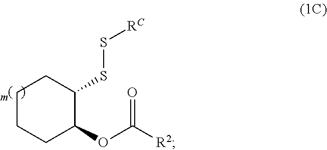

or a salt thereof; and
d) reacting a compound of Formula (1C), or a salt thereof, with R¹H to provide a compound of Formula (I).

In some embodiments, the compound of Formula (A-I) has Formula (A-I)′:

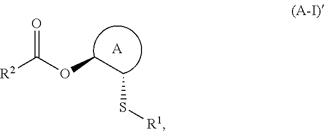

or a pharmaceutically acceptable salt thereof.

In some embodiments, m is 0. In some embodiments, m is 1. In some embodiments, m is 2.

In some embodiments, R^{C} is C₆₋₁₀ aryl or 5-10 membered heteroaryl. In some embodiments, R^{C} is 5-10 membered heteroaryl optionally substituted with 1, 2, 3, 4, or 5 substituents selected from C₁₋₄ alkyl, halo, CN, NO₂, OH, and OCH₃. In some embodiments, R^{C} is 5-10 membered heteroaryl. In some embodiments, R^{C} is pyridinyl. In some embodiments, R^{C} is pyridin-2-yl.

In some embodiments, R^{E} is C₆₋₁₀ aryl optionally substituted with 1, 2, 3, 4, or 5 substituents selected from C₁₋₄ alkyl, halo, CN, NO₂, OH, and OCH₃. In some embodiments, R^{E} is phenyl optionally substituted with 1, 2, 3, 4, or 5 substituents selected from C₁₋₄ alkyl, halo, CN, NO₂, OH, and OCH₃. In some embodiments, R^{E} is phenyl substituted with NO₂.

In some embodiments, R^{F} is halo. In some embodiments, R^{F} is chloro. In some embodiments, R^{F} is C₆₋₁₀ aryl optionally substituted with 1, 2, 3, 4, or 5 substituents selected from C₁₋₄ alkyl, halo, CN, NO₂, OH, and OCH₃. In some embodiments, R^{F} is phenyl optionally substituted with 1, 2, 3, 4, or 5 substituents selected from C₁₋₄ alkyl, halo, CN, NO₂, OH, and OCH₃. In some embodiments, R^{F} is phenyl substituted with NO₂.

In some embodiments, R^{C}—S—S—R^{C} is bis(5-nitrophenyl) carbonate.

Provided herein is a process for preparing a compound of Formula (I):

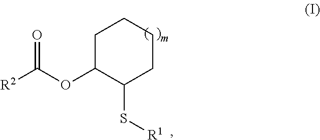

or a pharmaceutically acceptable salt thereof, wherein
R¹ is a targeting moiety; and
R² is a therapeutic moiety;
   comprising:
a) reacting a compound of Formula (1), or a salt thereof, which is prepared by a process disclosed herein, with R^{C}—S—S—R^{C} to provide a compound of Formula (8)

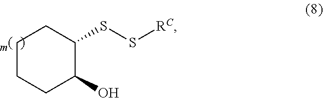

or a salt thereof, wherein R^{C} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents independently selected from C₁₋₄ alkyl, halo, CN, NO₂, OH, and OCH₃;
b) reacting Compound 1A, or a salt thereof, with bis(4-nitrophenyl) carbonate to provide Compound 1B having the formula:

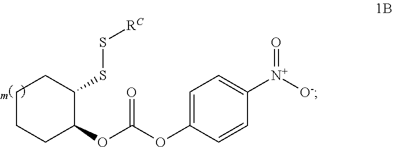

or a salt thereof;
c) reacting Compound 1B, or a salt thereof, with R²H to provide Compound 1C having the formula:

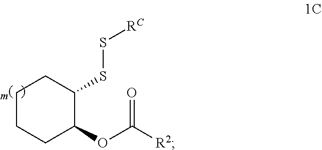

or a salt thereof;
d) reacting Compound 1C, or a salt thereof, with R¹H to provide a compound of Formula (I).

The targeting moiety can have affinity for a particular cell or tissue type where the presence of abnormal levels of a biomarker may be associated with one or more particular disease states. Typical biomarkers include cell surface proteins (e.g., receptors) including, but not limited to, the transferrin receptor; LDL receptor; growth factor receptors such as epidermal growth factor receptor family members (e.g., EGFR, Her2, Her3, Her4) or vascular endothelial growth factor receptors, cytokine receptors, cell adhesion molecules, integrins, selectins, and CD molecules. The marker can be a molecule that is present exclusively or in higher amounts on a malignant cell, e.g., a tumor antigen. In some embodiments, the targeting moiety is an antibody, or antibody fragment, that has specificity for an antigen expressed on a target cell, or at a target site, of interest. A wide variety of tumor-specific or other disease-specific antigens have been identified and antibodies to those antigens have been used or proposed for use in the treatment of such tumors or other diseases. The antibodies that are known in the art can be used in the compounds of the invention, in particular for the treatment of the disease with which the target antigen is associated.

In some embodiments, the targeting moiety is an antibody, antibody fragment, bispecific antibody or other antibody-based molecule or compound. In further embodiments, the targeting moiety can be an aptamer, avimer, receptor-binding ligand, nucleic acid, biotin-avidin binding pair, and the like.

In some embodiments, the targeting moiety is a peptide. In some embodiments, the peptide has 10 to 50 amino acids, 20 to 40 amino acids, 10 to 20 amino acids, 20 to 30 amino acids, or 30 to 40 amino acids.

In some embodiments, the targeting moiety is a conformationally restricted peptide. A conformationally restricted peptide can include, for example, macrocyclic peptides and stapled peptides. A stapled peptide is a peptide constrained by a covalent linkage between two amino acid side-chains, forming a peptide macrocycle. Conformationally restricted peptides are described, for example, in Guerlavais et al., Annual Reports in Medicinal Chemistry 2014, 49, 331-345; Chang et al., Proceedings of the National Academy of Sciences of the United States of America (2013), 110(36), E3445-E3454; Tesauro et al., Molecules 2019, 24, 351-377; Dougherty et al., Journal of Medicinal Chemistry (2019), 62(22), 10098-10107; and Dougherty et al., Chemical Reviews (2019), 119(17), 10241-10287, each of which is incorporated herein by reference in its entirety.

In some embodiments, the targeting moiety is an environmentally sensitive peptide described, for example, in U.S. Pat. Nos. 8,076,451 and 9,289,508 and U.S. Pat. Pub. No. 2019/209580 (each of which are incorporated herein by reference in their entirety), although other peptides capable of such selective insertion could be used. Other suitable peptides are described, for example, in Weerakkody, et al., PNAS 110 (15), 5834-5839 (Apr. 9, 2013), which is also incorporated herein by reference in its entirety. Without being bound by theory, it is believed that the environmentally sensitive peptide undergoes a conformational change and inserts across cell membranes in response to physiological changes (e.g., pH). The peptide can target acidic tissue and selectively translocate polar, cell-impermeable molecules across cell membranes in response to low extracellular pH. In some embodiments, the peptide is capable of selectively delivering molecules across a cell membrane having an acidic or hypoxic mantle having a pH less than about 6.0. In some embodiments, the peptide is capable of selectively delivering a molecule across a cell membrane having an acidic or hypoxic mantle having a pH less than about 6.5. In some embodiments, the peptide is capable of selectively delivering a molecule across a cell membrane having an acidic or hypoxic mantle having a pH less than about 5.5. In some embodiments, the peptide is capable of selectively delivering a molecule across a cell membrane having an acidic or hypoxic mantle having a pH between about 5.0 and about 6.0.

The term “acidic and/or hypoxic mantle” refers to the environment of the cell in the diseased tissue in question having a pH lower than 7.0 and preferably lower than 6.5. An acidic or hypoxic mantle more preferably has a pH of about 5.5 and most preferably has a pH of about 5.0. The compounds of formula (I) insert across a cell membrane having an acidic and/or hypoxic mantle in a pH dependent fashion to insert R²— into the cell, whereupon the disulfide linker is cleaved to deliver free R²H. Since the compounds of formula (I) are pH-dependent, they preferentially insert across a cell membrane only in the presence of an acidic or hypoxic mantle surrounding the cell and not across the cell membrane of “normal” cells, which do not have an acidic or hypoxic mantle. An example of a cell having an acidic or hypoxic mantle is a cancer cell.

The terms “pH-sensitive” or “pH-dependent” as used herein to refer to the peptide R¹ or to the mode of insertion of the peptide R¹ or of the compounds of the invention across a cell membrane, means that the peptide has a higher affinity to a cell membrane lipid bilayer having an acidic or hypoxic mantle than a membrane lipid bilayer at neutral pH. Thus, the compounds of the invention preferentially insert through the cell membrane to insert R²— to the interior of the cell (and thus deliver R²H as described above) when the cell membrane lipid bilayer has an acidic or hypoxic mantle (a “diseased” cell) but does not insert through a cell membrane when the mantle (the environment of the cell membrane lipid bilayer) is not acidic or hypoxic (a “normal” cell). It is believed that this preferential insertion is achieved as a result of the peptide R¹ forming a helical configuration, which facilitates membrane insertion.

In some embodiments, the environmentally sensitive peptide comprises at least one of the following sequences:

(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG;
(SEQ ID NO. 3; Pv3)
ADDQNPWRAYLDLLFPTDTLLLDLLWDADECG;
(SEQ ID NO. 4; Pv4)
Ac-AAEQNPIYWARYADWLFTTPLLLLDLALLVDADEGTKCG;
and
(SEQ ID No. 5; Pv5)
AAEQNPIYWARYADWLFTTPLLLLDLALLVDADEGTC.

In some embodiments, the environmentally sensitive peptide comprises at least one of the following sequences:

(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG,
and
(SEQ ID NO. 3; Pv3)
ADDQNPWRAYLDLLFPTDTLLLDLLWDADECG.

In some embodiments, the environmentally sensitive peptide comprises the sequence

(SEQ ID NO. 1; Pv1)
ADDQNPWRAYLDLLFPTDTLLLDLLWCG.

In some embodiments, the environmentally sensitive peptide comprises the sequence

(SEQ ID NO. 2; Pv2)
AEQNPIYWARYADWLFTTPLLLLDLALLVDADECG.

In some embodiments, the environmentally sensitive peptide comprises the sequence

(SEQ ID NO. 3; Pv3)
ADDQNPWRAYLDLLFPTDTLLLDLLWDADECG.

In some embodiments, the environmentally sensitive peptide comprises the sequence

(SEQ ID NO. 4; Pv4)
Ac-AAEQNPIYWARYADWLFTTPLLLLDLALLVDADEGTKCG.

In some embodiments, the environmentally sensitive peptide comprises the sequence

(SEQ ID NO. 5; Pv5)
AAEQNPIYWARYADWLFTTPLLLLDLALLVDADEGTC.

In some embodiments, the environmentally sensitive peptide consists essentially of the sequence ADDQNPWRAYLDLLFPTDTLLLDLLWCG (SEQ ID NO. 1; Pv1).

In some embodiments, the environmentally sensitive peptide consists essentially of the sequence AEQNPIYWARYADWLFTTPLLLLDLALLVDADECG (SEQ ID NO. 2; Pv2).

In some embodiments, the environmentally sensitive peptide consists essentially of the sequence ADDQNPWRAYLDLLFPTDTLLLDLLWDADECG (SEQ ID NO. 3; Pv3).

In some embodiments, the environmentally sensitive peptide consists essentially of the sequence AAEQNPIYWARYADWLFTTPLLLLDLALLVDADEGTKCG (SEQ ID NO. 4; Pv4).

In some embodiments, the environmentally sensitive peptide consists essentially of the sequence AAEQNPIYWARYADWLFTTPLLLLDLALLVDADEGTC (SEQ ID NO. 5; Pv5).

Additional environmentally sensitive peptides are disclosed in in U.S. Patent Publication No. US 2019/209580, U.S. patent application Ser. No. 16/925,094, and U.S. patent application Ser. No. 16/924,445, each of which is incorporated herein in its entirety.

The term “therapeutic moiety” refers to a moiety (e.g., R²—) derived from a therapeutic molecule or agent. Suitable therapeutic molecules (e.g., R²H) for use in the invention include PARP inhibitors, topoisomerase I inhibitors, and small molecule microtubule targeting moieties, which can have undesirable side effects when delivered systemically because of their possible deleterious effect on normal tissue.

Three PARP inhibitors (olaparib, rucaparib, and niraparib) are currently commercially available and others are in development, such as AG-014699 (Agouron/Pfizer), KU-0059436 (KuDOS/AstraZeneca), INO-1001 (Inotek/Genentech), NT-125 (now E-7449; Eisai; 3H-Pyridazino[3,4,5-de]quinazolin-3-one, 8-[(1,3-dihydro-2H-isoindol-2-yl)methyl]-1,2-dihydro-), 2X-121 (2X Oncology; 3H-pyridazino[3,4,5-de]quinazolin-3-one, 8-[(1,3-dihydro-2H-isoindol-2-yl)methyl]-1,2-dihydro-), and ABT-888 (Abbvie). PARP inhibitors are disclosed in (for example) U.S. Pat. Nos. 6,100,283; 6,310,082; 6,495,541; 6,548,494; 6,696,437; 7,151,102; 7,196,085; 7,449,464; 7,692,006; 7,781,596; 8,067,613; 8,071,623; and 8,697,736, which patents are incorporated herein by reference in their entirety.

Compounds of Formula (I) containing a PARP inhibitor moiety are described in U.S. Patent Publication No. US 2019/209580.

The term “small molecule topoisomerase I targeting moiety” or “topoisomerase I inhibitor” refers to a chemical group that binds to topoisomerase I. The small molecule topoisomerase I targeting moiety can be a group derived from a compound that inhibits the activity of topoisomerase I. Topoisomerase inhibitors include camptothecin and derivatives and analogues thereof such as opotecan, irinotecan (CPT-11), silatecan (DB-67, AR-67), cositecan (BNP-1350), lurtotecan, gimatecan (ST1481), belotecan (CKD-602), rubitecan, topotecan, deruxtecan, and exatecan. Topoisomerase inhibitors are described in, for example, Ogitani, Bioorg. Med. Chem. Lett. 26 (2016), 5069-5072; Kumazawa, E., Cancer Chemother Pharmacol 1998, 42: 210-220; Tahara, M, Mol Cancer Ther 2014, 13(5): 1170-1180; Nakada, T., Bioorganic & Medicinal Chemistry Letters 2016, 26: 1542-1545.

Compounds of Formula (I) having a topoisomerase I targeting moiety are described in U.S. patent application Ser. No. 16/925,094. In some embodiments of compounds of Formula (I), R² is camptothecin, opotecan, irinotecan (CPT-11), silatecan (DB-67, AR-67), cositecan (BNP-1350), lurtotecan, gimatecan (ST1481), belotecan (CKD-602), rubitecan, topotecan, deruxtecan, or exatecan. In some embodiments of compounds of Formula (I), R² is exatecan.

Suitable small molecule microtubule targeting moieties (e.g., R²) can be cytotoxic compounds like maytansinoids that may have undesirable side effects when delivered systemically because of their possible deleterious effect on normal tissue. Small molecule microtubule targeting agents include, but are not limited to, maytansinoids, aclitaxel, docetaxel, epothilones, discodermolide, the vinca alkaloids, colchicine, combretastatins, and derivatives and analogues of the aforementioned. Microtubule targeting agents are described in Tangutur, A. D., Current Topics in Medicinal Chemistry, 2017 17(22): 2523-2537. Microtubule-targeting agents also include maytansinoids, such as maytansine (DM1) and derivatives and analogues thereof, which are described in Lopus, M, Cancer Lett., 2011, 307(2): 113-118; and Widdison, W., J. Med. Chem. 2006, 49:4392-4408.

Compounds of Formula (I) having a microtubule targeting moiety are described in U.S. patent application Ser. No. 16/924,445. In some embodiments, R² is a maytansinoid. In some embodiments, R² is DM1 or DM4. In some embodiments, R² is DM1. In some embodiments, R² is DM4.

In some embodiments, the compound of formula (I) is selected from:

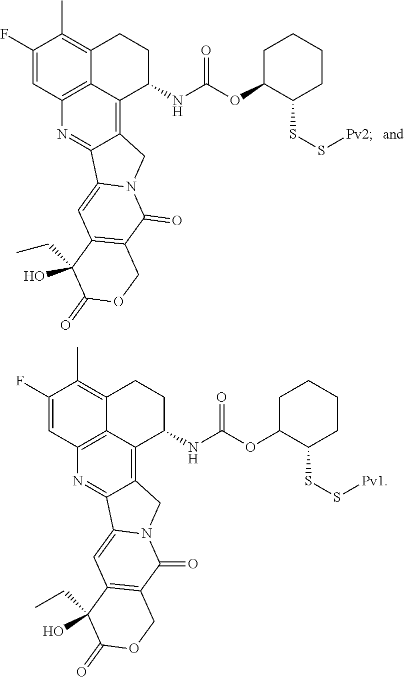

The molecules of the invention can be tagged, for example, with a probe such as a fluorophore, radioisotope, and the like. In some embodiments, the probe is a fluorescent probe, such as LICOR. A fluorescent probe can include any moiety that can re-emit light upon light excitation (e.g., a fluorophore).

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula (I) can be combined in any suitable combination.

As used herein, “about” means ±20% of the stated value, and includes more specifically values of ±10%, ±5%, ±2% and ±1% of the stated value.

As used herein, the term “reacting,” or “contacting” when describing a certain process is used as known in the art and generally refers to the bringing together of chemical reagents in such a manner so as to allow their interaction at the molecular level to achieve a chemical or physical transformation. In some embodiments, the reacting involves two reagents, wherein one or more equivalents of second reagent are used with respect to the first reagent. The reacting steps of the processes described herein can be conducted for a time and under conditions suitable for preparing the identified product.

The term “base” refers to a compound that is an electron pair donor in an acid-base reaction.

The term “acid” refers to a compound that is an electron pair acceptor in an acid-base reaction.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

Suitable solvents can include halogenated solvents such as carbon tetrachloride, bromodichloromethane, dibromochloromethane, bromoform, chloroform, bromochloromethane, dibromomethane, butyl chloride, dichloromethane (methylene chloride), tetrachloroethylene, trichloroethylene, 1,1,1-trichloroethane, 1,1,2-trichloroethane, 1,1-dichloroethane, 2-chloropropane, 1,1,1-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

Suitable ether solvents include: dimethoxymethane, tetrahydrofuran, cyclopentyl methyl ether, 1,3-dioxane, 1,4-dioxane, furan, tetrahydrofuran (THF), diethyl ether, ethylene glycol dimethyl ether, ethylene glycol diethyl ether, diethylene glycol dimethyl ether (diglyme), diethylene glycol diethyl ether, triethylene glycol dimethyl ether, anisole, methyl tert-butyl ether, mixtures thereof and the like.

The term “acylating reagent” refers to a compound that contributes a carbonyl group to a nucleophilic position of a reactant compound. For example, an electrophilic carbonyl group can react with a nucleophilic O or N atom. Exemplary acylating reagents include isopropenyl acetate, succinic anhydride, and glutaric anhydride.

The term “reducing agent” refers to a compound that contributes a hydride to an electrophilic position of a reactant compound such as an unsaturated carbon (e.g. carbon of a carbonyl moiety or imine moiety). For example, the reducing agent can contributes a hydride to a reactant compound converting an amide containing reactant compound to an amine product compound, converting an imine containing reactant compound to an amine product compound, converting a ketone containing reactant compound to an alcohol product compound or converting an ester containing reactant compound to an alcohol product compound.

The reactions of the processes described herein can be carried out in air or under an inert atmosphere. Typically, reactions containing reagents or products that are substantially reactive with air can be carried out using air-sensitive synthetic techniques that are well known to the skilled artisan.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as high performance liquid chromatography (HPLC) or thin layer chromatography. The compounds obtained by the reactions can be purified by any suitable method known in the art. For example, chromatography (medium pressure) on a suitable adsorbent (e.g., silica gel, alumina and the like), HPLC, or preparative thin layer chromatography; distillation; sublimation, trituration, or recrystallization. The purity of the compounds, in general, are determined by physical methods such as measuring the melting point (in case of a solid), obtaining a NMR spectrum, or performing a HPLC separation. If the melting point decreases, if unwanted signals in the NMR spectrum are decreased, or if extraneous peaks in an HPLC trace are removed, the compound can be said to have been purified. In some embodiments, the compounds are substantially purified.

The expressions, “ambient temperature” and “room temperature,” as used herein, are understood in the art, and refer generally to a temperature, e.g., a reaction temperature, that is about the temperature of the room in which the reaction is carried out, for example, a temperature from about 20° C. to about 30° C.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene”, employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The terms “halo” or “halogen”, used alone or in combination with other terms, refers to fluoro, chloro, bromo and iodo. In some embodiments, “halo” refers to a halogen atom selected from F, Cl, or Br. In some embodiments, halo groups are F.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “ Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl.

The term “heteroaryl” or “heteroaromatic,” employed alone or in combination with other terms, refers to a monocyclic or polycyclic aromatic heterocycle having at least one heteroatom ring member independently selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopentyl, cyclohexyl, and cycloheptyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include tetrahydropyranyl.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The terms “protecting” and “deprotecting” as used herein in a chemical reaction refer to inclusion of a chemical group in a process and such group is removed in a later step in the process. The term preparation of Compound 1 and its salts can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006). Examples of protecting groups include thio-protecting groups.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### EXAMPLES

As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

### Starting Materials & Enzymes

Enzymes were obtained from Novozymes, Genencor, Sigma-Aldrich (including the Amano enzymes), c-Lecta, Aum Enzymes and immobilized on Immobead COV-1. Starting materials were obtained from TCI and Sigma-Aldrich.

### Chiral Gas Chromatography (GC)

Chiral GC was conducted using a Supelco betaDEX 325 (30 m×0.25 mm×0.25 μm df),

using hydrogen as carrier gas at a linear velocity of 0.5 m/sec and temperature gradient operation (2-10° C./min rate).

The (S,S)-enantiomer of 2-(acetylthio)cyclohexyl acetate eluted first. Other components were not resolved on chiral GC using this column.

Retention times:

| Product | T(° C.) | t_{R} (min; R/S) | Remarks | |
|---|---|---|---|---|
| 2-Mercaptocyclohexanol | 50-210 | 9.9 | 10° | C./min |
| | 120-210 | 3.79 | 10° | C./min |
| | 120-160 | 5.8 | 2° | C./min |
| 2-mercaptocyclohexyl | 120-210 | 4.7 | 10° | C./min |
| acetate | 120-160 | 7.8 | 2° | C./min |
| 2-(acetylthio)cyclohexyl | 120-210 | 7.3 | 10° | C./min |
| acetate | 120-160 | 15.7/15.5 | 2° | C./min |
| Benzylthiocyclohexanol | 50-210 | 20.0 | 10° | C./min |

### Achiral GC

Achiral GC was used for several conversion determinations and for samples incompatible with the sensitive chiral column. A Supelco METbiodiesel column was used with hydrogen as carrier gas at linear velocity of 0.5 m/s. Fast gradient of 50-250° C. was used at 20° C./min.

### Retention Times:

| Product | T(° C.) | t_{R} (min) | Remarks |
|---|---|---|---|
| 2-Mercaptocyclohexanol | 50-250 | 1.55 | cis-isomer 1.42 min |
| Benzylthiocyclohexanol | 50-250 | 6.0 | |
| 2-Mercaptopyridine | 50-250 | 2.5 | broad |
| Dipyridyldisulfide | 50-250 | 6.8 | |
| 2-(pyridin-2- | 50-250 | 7.2 | |
| yldisulfanyl)cyclohexan-1-ol | | | |
| 2-(pyridin-2- | 50-250 | 7.8 | |
| yldisulfanyl)cyclohexyl acetate | | | |

### Chiral HPLC

Chiral HPLC was performed using a ChiralPak AD3 column 250×4.6 mm, using a heptane/isopropanol/ethanol mixture. The benzyl thioether derivatives could be analyzed using heptane/isopropanol 90/10 mixture (eluent A), the more polar pyridyldisulfide derivatives required heptane/isopropanol/ethanol 63/7/30 (eluent B). Detection was performed at 220 nm.

### Retention Times:

| Product | Eluent | t_{R} (min; R/S) | Remarks |
|---|---|---|---|
| Benzyl mercaptan | A | 3.9 | |
| Benzylthiocyclohexanol | A | 8.7/9.2 | |
| Benzylthiocyclohexyl acetate | A | 5.1 | Not resolved |
| Benzylthiocyclohexyl glutarate | A | 10.2/14.3 | Second peak not |
| | | | determined |
| 2-(pyridin-2-yldisulfanyl)cyclohexan-1-ol | B | 6.4/14.7 | |
| 2-(pyridin-2-yldisulfanyl)cyclohexyl acetate | B | 7.3/5.4 | |
| 4-oxo-4-((2-(pyridin-2- | B | 9.9/12.7 | |
| yldisulfanyl)cyclohexyl)oxy)butanoic | | | |
| acid | | | |
| 5-oxo-5-((2-(pyridin-2- | B | 10.2/11.5 | |
| yldisulfanyl)cyclohexyl)oxy)pentanoic | | | |
| acid | | | |
| 2-Mercaptopyridine | B | 7.1 | Broad |
| Dipyridyldisulfide | B | 7.1 | Sharp |

### Example 1. Synthesis of Zinc (D)-tartrate

(D)-Tartaric acid (150 g; 1 mol) was dissolved in water (1.5 L). A solution of zinc chloride (136 g; 1 mol) in water (1 L) and 20 ml 3.5% HCl (20 mL) was added under mechanical stirring. The acidic mixture was neutralized toward pH 4 using 33% aqueous sodium hydroxide. A precipitate was formed and the mixture was stirred for another hour. The mixture was filtered on a P3 glass filter. The solid was washed with water, acetone and ethyl acetate. The solid was dried in a vacuum oven to yield the desired product as a white, high density powder (270 g).

Alternatively, (D)-Tartaric acid (15 g; 0.1 mol) dissolved in deionized water (0.15 L) was neutralized to pH 12 using 33% NaOH (20 ml; 0.2 mol). A solution of zinc chloride (13.6 g) in deionized water (50 mL) was added dropwise under mechanical stirring. Initially, a gel was formed, which transformed into milky suspension. At the end of addition, the pH had dropped to neutral. Filtration was performed using a P3 glass filter (over about 1 h), and the solid was washed with deionized water, acetone and ethyl acetate. Vacuum drying in an oven yielded a white, fluffy powder (22 g).

### Example 2. Synthesis of Racemic 2-benzylthiocyclohexanol

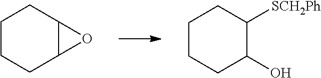

Benzyl mercaptan (1.24 g; 10 mmol) and cyclohexene oxide (1.9 g; 19 mmol) were dissolved in 2-methyltetrahydrofuran. N-Methyl morpholine (0.5 ml) was added. No reaction occurred overnight. Addition of sodium ethoxide solution (1 ml 20%) gave quick reaction (50% in 1 h, complete conversion of mercaptan). Aqueous workup and evaporation yielded a yellow oil (2.18 g). The oil was distilled under reduced pressure using a Kugelrohr apparatus (oven 145° C./<1 mbar) yielding a clear oil (1.89 g; 8.5 mmol; 85%). HPLC: 2 peaks 8.7 m (R,R) and 9.2 m (S,S) using Chiralpak AD3 with heptane/isopropanol 90/10. UV detection at 220 nm.

### Example 3. Enriched 2-benzylthiocyclohexanol

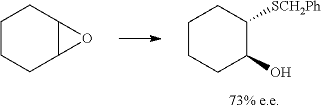

A 5 L reactor was charged with zinc (D)-tartrate (263 g), dichloromethane (2 L), benzylmercaptan (124 g; 1 mol) and cyclohexene oxide (147 g; 1.5 eq.). The reaction was placed under an Ar atmosphere. The mixture was stirred for 5 days (resulting in >99% mercaptan conversion) and filtered after 7 days. The filtrate was evaporated and transferred to a smaller flask using ethyl acetate. Evaporation and high vacuum distillation yielded a clear, colourless oil (215 g). HPLC: 87% (S,S) [75% e.e] and 1% other components.

The zinc (D)-tartrate catalyst could be reused. A 2 L reactor was charged with recovered zinc (D)-tartrate catalyst (468 g) dichloromethane (1 L), benzylmercaptan (124 g; 1 mol) and cyclohexene oxide (147 g; 1.5 eq.). The reaction was placed under an Ar atmosphere. The mixture was stirred for 1 day (resulting >99% mercaptan conversion) and filtered. The filtrate was evaporated and transferred to a smaller flask using ethyl acetate. Evaporation yielded a clear, colourless oil (225 g). HPLC: 85% (S,S) [73% e.e] and 1% other components.

### Example 4. Enzyme Screening Experiment

An amount of 20-25 mg of enzyme was added to a 2 mL vial. To this was added racemic 2-benzylthiocyclohexanol (22 mg), dissolved in 1 mL anhydrous MTBE containing 5 vol % isopropenyl acetate. The vial was closed and shaken at 24° C. for 16 h in a thermostatted shaker. After the incubation, analysis by chiral HPLC was performed using Chiralpak AD3, 100× dil in mobile phase; heptane/isopropanol (90/10). Results of the screen are shown in the table below.

| 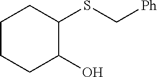 [SMILES: OC1CCCCC1SCc1ccccc1] | | 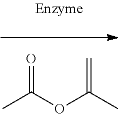 | 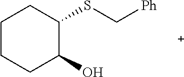 [SMILES: O[C@H]1CCCC[C@@H]1SCc1ccccc1] | | 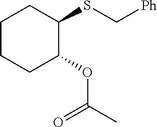 [SMILES: CC(=O)O[C@@H]1CCCC[C@H]1SCc1ccccc1] |
|---|---|---|---|---|---|
| Entry | Enzyme | Source | Conv | Substrate ee | E (est) |
| 1 | IMMCALA-T2- | Candida antarctica, | 21% | 6.6% | <2 |
| | 150 | A | | | |
| 2 | IMMCALB-T2- | Candida antarctica, | 49% | >99.8% | >1000 |
| | 150 | B | | | |
| 3 | IMMCALBY- | Candida antarctica, | 49% | >99.8% | >1000 |
| | T2-150 | B | | | |
| 4 | IMMRML-T2- | Rhizomucor miehei | 2% | | |
| | 150 | | | | |
| 5 | IMMTLL-T2- | Thermomyces | 47% | 91.3% | >500 |
| | 150 | lanuginosa | | | |
| 6 | IMMCRL-T2- | Candida rugosa | 7% | | |
| | 150 | | | | |
| 7 | IMMABC-T2- | Pseudomonas | 48% | 96.5% | >500 |
| | 150 | cepacia | | | |
| 8 | IMMAPF-T2- | Pseudomonas | 49% | 98.9% | >500 |
| | 150 | fluorescens | | | |
| 9 | IMMARO-T2- | Rhizopus oryzae | 16% | 20% | |
| | 150 | | | | |
| 10 | IMMAMJ-T2- | Mucor javanicus | 12% | 16% | |
| | 150 | | | | |
| 11 | IMMANA-T2- | Aspergillus niger | 7% | | |
| | 150 | | | | |
| 12 | IMMRNA-T2- | Rhizopus niveus | <1% | | |
| | 150 | | | | |
| 13 | IMMASMQ-T2- | Alcaligenes sp. | 8% | | |
| | 150 | | | | |
| 14 | IMMRES-T2- | Resinase HT | 49% | 99.3% | >500 |
| | 150 | | | | |
| 15 | IMMLIPX-T2- | Lipex 100L | 49% | 99.5% | >1000 |
| | 150 | | | | |
| 16 | IMML51-T2- | Novozymes | 50% | 99.1% | >300 |
| | 150 | Stickaway | | | |
| 17 | IMMCCMO-T2- | Candida cylindracea | 7% | | |
| | 150 | sp. | | | |
| 18 | IMMAULI-T2- | Bacillus subtilis | 30% | 44% | >100 |
| | 150 | | | | |

Further studies employing cyclic anhydrides (e.g., glutaric anhydride and succinic anhydride) were also effective.

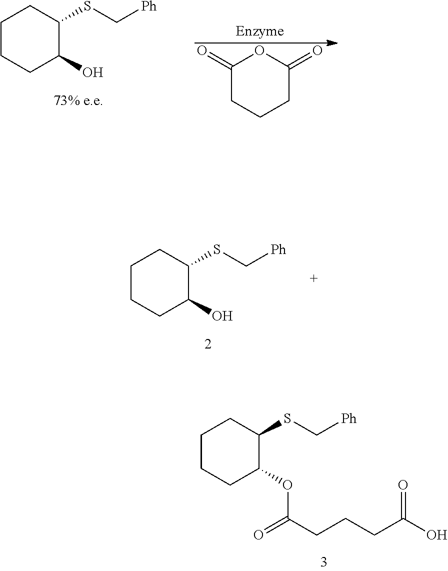 [SMILES: O[C@H]1CCCC[C@@H]1SCc1ccccc1 ; O=C1CCCC(=O)O1 ; O[C@H]1CCCC[C@@H]1SCc1ccccc1 ; O=C(O)CCCC(=O)O[C@@H]1CCCC[C@H]1SCc1ccccc1]

Glutaric anhydride was used in place of isopropenyl acetate in the above enzyme screening experiment. The use of the enzyme CaL-B-T2 allowed for R-selective removal to provide an 85:15 mixture of Compound 2 and Compound 3. Compound 3 was removed from the reaction mixture via base extraction with 2 M NH₃ followed by a mild base wash (e.g., aqueous Na₂CO₃). The product was dried under Ar to yield Compound 2.

### Example 5. Enzymatic Resolution of Enriched 2-Benzylthiocyclohexanol

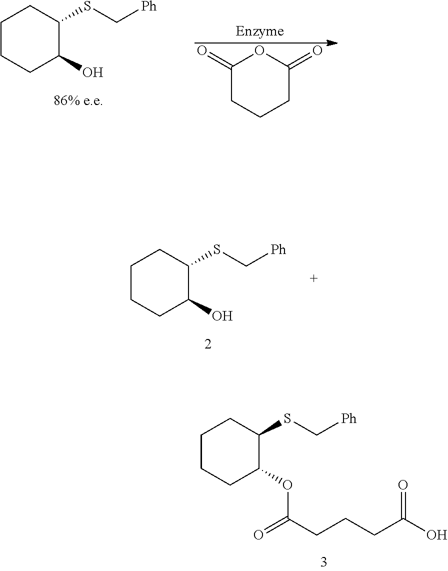 [SMILES: O[C@H]1CCCC[C@@H]1SCc1ccccc1 ; O=C1CCCC(=O)O1 ; O[C@H]1CCCC[C@@H]1SCc1ccccc1 ; O=C(O)CCCC(=O)O[C@@H]1CCCC[C@H]1SCc1ccccc1]

Enriched 2-benzylthiocyclohexanol (225 g; 86% e.e.; 1 mol) was placed in a 2 L flask. MTBE (1.5 L), glutaric anhydride (33 g; 0.29 mol) and immobilized enzyme (Product No. CaLB-ADS4 from ChiralVision; 50 g) were added. The mixture was stirred at 180 rpm using a mechanical stirrer for 2 days. After 1 day the minor (R,R)-enantiomer was completely removed. Next day the reaction mixture was filtered and the enzyme washed with isopropyl acetate. The filtrate was washed with a 2 M solution of aqueous ammonia (0.5 L) and 1.25 M aqueous solution of sodium carbonate (0.5 L). The organic phase was isolated, dried on sodium sulfate and evaporated to provide Compound 2 as a slightly turbid colorless oil (172 g; 76%).

HPLC: 98.0%, 100.0% e.e.. About 1.1% of dibenzyldisulfide was detected.

### Example 6. Reductive Cleavage of Compound 2

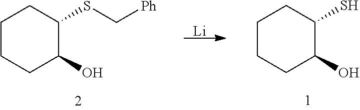

10 Distilled (S,S)-2-benzylthiocyclohexanol (11.1 g; 50 mmol) was placed in a dry 250 mL round bottom flask under argon atmosphere and dissolved in 100 mL anhydrous 2-methyltetrahydrofuran. Under mechanical stirring, lithium grains (1.4 g total; 200 mmol) were added in 2 portions. The reaction was cooled in a water bath. Overnight stirring at ambient temperature yielded a grey slurry. This slurry, containing unreacted excess lithium, was poured into 100 ml cold water. After complete quench of the lithium metal, the clear solution was phase separated. The aqueous phase containing the lithium salt of the desired product (pH>11) was acidified with solid citric acid (10 g) to pH 5. Extraction with 100 mL ethyl acetate, drying on sodium sulfate and careful evaporation yielded (S,S)-2-mercaptocyclohexanol as a light yellow oil (5 g; 38 mmol; 76%. GC: >99%. [α]_{D}: +42° (c=1 in MeOH)).

### Example 7. (S,S)-2-Pyridin-2-yldisulfaneyl)cyclohexanol

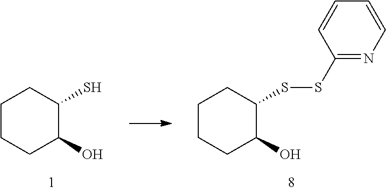 [SMILES: O[C@H]1CCCC[C@@H]1S ; O[C@H]1CCCC[C@@H]1SSc1ccccn1]

(S,S)-2-Mercaptocyclohexanol (7.0 g; 53 mmol) was dissolved in 50 mL methanol under argon and added dropwise to a solution of dipyridyldisulfide (12 g; 55 mmol) in methanol (100 mL). After 1.5 hour, the reaction mixture was evaporated to dryness and the residue mixed with MTBE (100 mL). The precipitated 2-mercaptopyridine was removed by filtration and the clear filtrate washed with 1 M sodium carbonate solution (2× 100 mL), dried on sodium sulfate and evaporated to a yellow oil (13 g). The oil was triturated with 100 ml n-heptane to a light brown solid (11 g (86%); GC: 91%).

The solid was purified by dissolution in MTBE (25 mL), mixing with 50 mL heptane and seeded with the desired product (seeds were obtained from, for example, Step 1 of Example 9). A white crystalline powder was formed. The mixture was cooled in an ice bath and filtered. The solid was washed with n-heptane (25 ml). Careful vacuum drying yielded a white powder (7.6 g; 31.5 mmol; 59% yield from mercaptocyclohexanol; 44% yield from 2-benzylthiocyclohexanol). GC: 99.0%; HPLC: 99.8%; Chiral HPLC: 100% e.e.; mp: 70-72° C.; [α]_{D}: −146° (c=1 in MeOH).

### Example 8. 4-Nitrophenyl (S,S)-2-pyridin-2-yldisulfaneyl)cyclohexyl) Carbonate

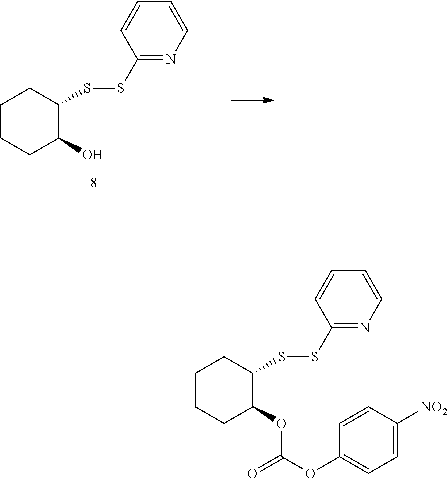 [SMILES: O[C@H]1CCCC[C@@H]1SSc1ccccn1 ; O=C(Oc1ccc([N+](=O)[O-])cc1)O[C@H]1CCCC[C@@H]1SSc1ccccn1]

(S,S)-2-Pyridin-2-yldisulfaneyl)cyclohexanol (4.8 g; 20 mmol) was placed in a dry flask under argon atmosphere and dissolved in 80 mL anhydrous dichloromethane. Pyridine (5 mL; 60 mmol; 3 eq.) was added. A solution of 4-nitrophenyl chloroformate (4.08 g; 20.2 mmol) in 40 mL anhydrous dichloromethane was added dropwise under argon in about 1 hour at ambient temperature. HPLC sample showed 2% residual (S,S)-2-pyridin-2-yldisulfaneyl)cyclohexanol, 3% bis(4-nitrophenyl) carbonate and 95% desired product. Further stirring for 1 hour did not increase conversion. The mixture was quenched with water (10 mL) and washed with 0.5 M aqueous hydrochloric acid (100 mL), saturated aqueous sodium bicarbonate (25 mL) and dried on sodium sulfate. Evaporation yielded a yellow oil (8.4 g). This oil was dissolved in a mixture of MTBE (35 mL) and n-heptane (50 mL). Heating to 50° C. and slow cooling to 30° C. under agitation and seeding yielded a thick suspension. This was further diluted with n-heptane (20 mL) and cooled in an ice bath. The precipitate was filtered, washed with n-heptane (30 mL) and allowed to dry under vacuum to yield the desired product as a white powder (16.3 mmol; 81%).

HPLC: 99.4%; Chiral HPLC: 99.2% purity, 100% e.e.; mp: 77-79° C.; [α]_{D}: +104° (c=1 in MeOH).

### Example 9. Synthesis of Conjugate 1

Conjugate 1 can be prepared according to the process disclosed in Example 11 of U.S. patent application Ser. No. 16/925,094, using Compound 1 ((1S,2S)-2-mercaptocyclohexan-1-ol) in place of racemic 2-mercaptocyclohexan-1-ol. Example 11 of U.S. patent application Ser. No. 16/925,094 is reproduced below.

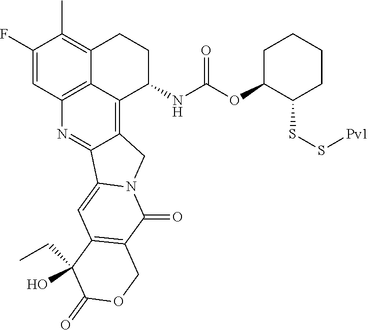

### Step 1. Synthesis of 2-(pyridine-2-yldisulfanyl)cyclohexan-1-ol

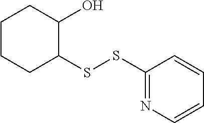 [SMILES: OC1CCCCC1SSc1ccccn1]

To a solution of 1,2-di(pyridine-2-yl)disulfane (15.2 g, 68.9 mmol) in MeOH (degassed with N₂) (30 mL) was added 2-mercaptocyclohexan-1-ol (11.4 g, 86.2 mmol) (degassed with N₂) dropwise and stirred for 16 h at room temperature under an N₂ atmosphere. The reaction mixture was concentrated to dryness under vacuum. The resultant crude material was purified by column chromatography using 30% EtOAC/hexanes to afford the title compound as a yellow liquid. ¹HNMR (400 MHz, CDCl₃): δ 8.54-8.53 (m, 1H), 7.60-7.56 (m, 1H), 7.40-7.38 (m, 1H), 7.17-7.14 (m, 1H), 3.38-3.34 (m, 1H), 2.62-2.57 (m, 1H), 2.11-2.02 (m, 1H), 1.75-1.74 (m, 2H), 1.61-1.60 (m, 1H), 1.42-1.24 (m, 4H).

The title compound was subjected to chiral preparative HPLC conditions (Chiralpak IG: 250 mm×20 mm×5 mic; n-Hexane: IPA with 0.1% Diethylamine (80:20); 19 mL/min; 25° C. (Room Temperature). (1R,2R)-2-(pyridin-2-yldisulfanyl)cyclohexan-1-ol (4.5 g, 18.6 mmol) eluted first (retention time: 3.9 minutes), followed by (1S,2S)-2-(pyridin-2-yldisulfanyl)cyclohexan-1-ol (retention time: 11.3 minutes). The absolute stereochemistry was confirmed by comparison of the product of Step 2 with chiral material having a reported absolute stereochemistry (see Monaco, M. R.; J. Am. Chem. Soc. 2014, 136, 49, 16982-16985).

### Step 2. Synthesis of 4-nitrophenyl ((1S,2S)-2-(pyridin-2-yldisulfanyl)cyclohexyl) Carbonate.

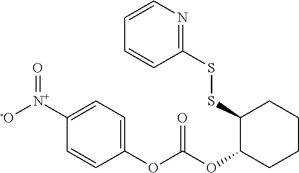

To a solution of (1R,2R)-2-(pyridin-2-yldisulfanyl)cyclohexan-1-ol (4.5 g, 18.6 mmol) in DMF (90.0 mL) was added DIPEA (10.3 mL, 56.0 mmol) and bis(4-nitrophenyl) carbonate (11.35 g, 27.3 mmol) at room temperature. The reaction vessel was sealed and stirred at room temperature for 12 h. Progress of the reaction was monitored by TLC (20% EtOAc/hexanes). After completion of the reaction, the reaction mixture was quenched with water (20.0 mL) and extracted with EtOAc (20.0 mL). The organic layer was separated, washed with brine, dried over anhydrous sodium sulfate and concentrated under reduced pressure to afford the crude product, which was purified by column chromatography using 20-30% EtOAc/hexanes to afford the title product as an off-white solid (5.0 g, 66% yield). ¹HNMR (400 MHz, CDCl₃): δ 8.44 (d, J=4 Hz, 1H), 8.28 (d, J=8.8 Hz, 2H), 7.72 (d, J=8.4 Hz, 1H), 7.61-7.57 (t, J=7.6 Hz, 1H), 7.41 (d, J=9.6 Hz, 2H), 7.08-7.05 (t, J=5.2 Hz, 1H), 4.85-4.74 (m, 1H), 3.03-2.92 (m, 1H), 2.28 (d, J=9.6 Hz, 1H), 2.20-2.12 (m, 1H), 1.85-1.62 (m, 3H), 1.45-1.25 (m, 3H). LC-MS m/z calculated: 406.7; found: 407.4 [M+H]⁺.

### Step 3. Synthesis of [(1S,2S)-2-(2-pyridyldisulfanyl)cyclohexyl]N-[(10S,23S)-10-ethyl-18-fluoro-10-hydroxy-19-methyl-5,9-dioxo-8-oxa-4,15-diazahexacyclo[14.7.1.02,14.04,13.06,11.020,24]tetracosa-1,6(11),12,14,16(24), 17,19-heptaen-23-yl]carbamate.

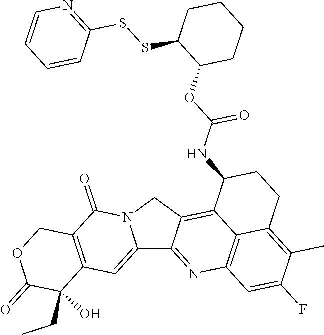 [SMILES: CC[C@@]1(O)C(=O)OCc2c1cc1n(c2=O)Cc2c-1nc1cc(F)c(C)c3c1c2[C@@H](NC(=O)O[C@H]1CCCC[C@@H]1SSc1ccccn1)CC3]

To (10S,23S)-23-amino-10-ethyl-18-fluoro-10-hydroxy-19-methyl-8-oxa-4,15-diazahexacyclo[14.7.1.02,14.04,13.06,11.020,24]tetracosa-1,6(11),12,14,16(24),17,19-heptaene-5,9-dione methanesulfonic acid (250 mg, 0.470 mmol) in 10 mL of dry DMF was added 4-nitrophenyl ((1S,2S)-2-(pyridin-2-yldisulfanyl)cyclohexyl) carbonate (from Step 2; 191 mg, 0.470 mmol), N,N-diisopropylethylamine (122 mg, 0.941 mmol) and DMAP (115 mg, 0.941 mmol). The mixture was stirred at room temperature overnight. LC-MS indicated that the desired coupling product had formed. The reaction mixture was then diluted with EtOAc, washed with saturated aqueous NH₄Cl, H₂O, and brine. The mixture was dried over sodium sulfate, filtered, and concentrated. The crude residue was purified by column chromatography using 0-5% MeOH/dichloromethane to give 240 mg of the desired product in 72.6% yield (240 mg).

### Step 4. Coupling with Pv1 (Compound 11)

In a vial was added Pv1 (275 mg, 0.0811 mmol), the compound of Step 3 (74.1 mg, 0.105 mmol), acetonitrile (10 mL) and water (5 mL). n-Methylmorpholine (0.303 g, 0.0030 mol) was added to this mixture. The mixture was stirred at room temperature overnight. LC-MS indicated that the desired coupled product had been formed.

The reaction mixture was purified directly by reverse phase HPLC (20-85% acetonitrile/water, 0.5% acetic acid on a Sunfire Prep C18 column (10 μm, 50×150 mm), retention time: 7.022 min) to give 213 mg of the desired product in 68% yield (213 mg). ESI (M+3H/3)³⁺: 1291.6

### Example 10: Enzyme Screen of Alternative Substrate

An amount of 20-25 mg of enzyme was added to a 2 mL vial. To this was added racemic 2-(pyridin-2-yldisulfanyl)cyclohexan-1-ol (12 mg), dissolved in 1 mL 2-methyltetrahydrofuran containing 5 vol % isopropenyl acetate. The vial was closed and shaken at 21° C. for two days in a thermostatted shaker. After the incubation, analysis by chiral HPLC was performed using Chiralpak AD3, 100× dil in mobile phase; heptane/isopropanol/ethanol (63/7/30). Results of the screen are shown in the table below.

| | | | |
|---|---|---|---|
| 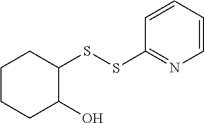 [SMILES: OC1CCCCC1SSc1ccccn1] | 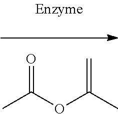 | 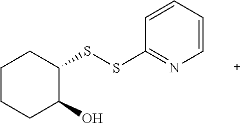 [SMILES: O[C@H]1CCCC[C@@H]1SSc1ccccn1] | 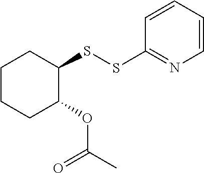 [SMILES: CC(=O)O[C@@H]1CCCC[C@H]1SSc1ccccn1] |

| | | | | Conv | Substrate | Product | |
|---|---|---|---|---|---|---|---|
| Entry | Enzyme | m(mg) | t(h) | (area) | e.e. | e.e. | E |
| 1 | IMMCALA-T2-150 | 22.3 | 42 | 7% | — | 8% | 1.2 |
| 2 | IMMCALB-T2-150 | 20.1 | 24 | 50% | 99.3% | >99.9% | |
| | | | 42 | 50.5% | >99.9% | >99.9% | >15000 |
| 3 | IMMCALBY-T2-150 | 20.1 | 42 | 49.1% | 93% | >99.8% | >5000 |
| 4 | IMMRML-T2-150 | 20.3 | 42 | 0% | | | |
| 5 | IMMTLL-T2-150 | 22.4 | 42 | 6% | | >99% | >200 |
| 6 | IMMCRL-T2-150 | 20.3 | 42 | 0% | | | |
| 7 | IMMABC-T2-150 | 22.7 | 42 | 8% | | >99% | >200 |
| 8 | IMMAPF-T2-150 | 20.5 | 42 | 12% | | >99% | >200 |
| 9 | IMMARO-T2-150 | 21.0 | 42 | 0% | | | |
| 10 | IMMAMJ-T2-150 | 22.0 | 42 | 0% | | | |
| 11 | IMMANA-T2-150 | 20.4 | 42 | 0% | | | |
| 12 | IMMRNA-T2-150 | 22.8 | 42 | 0% | | | |
| 13 | IMMASMQ-T2-150 | 23 | 42 | 1% | | | |
| 14 | IMMRES-T2-150 | 23.3 | 42 | 13% | | 87% | 16 |
| 15 | IMMLIPX-T2-150 | 20.7 | 42 | 19%* | | 93% | |
| 16 | IMML51-T2-150 | 22.3 | 42 | 39% | | 97% | 130 |
| 17 | IMMCCMO-T2-150 | 21.5 | 42 | 1% | | | |
| 18 | IMMAULI-T2-150 | 20.4 | 42 | 1% | | | |

Enantioenriched 2-(pyridine-2-yldisulfanyl)cyclohexan-1-ol can be used in place of racemic material in Example 2 Step 6 in order to provide Conjugate 1 of Example 9.

### Example 11: Synthesis of 4-Nitrophenyl[(S,S)-2-(pyridin-2-yldisulfaneyl)cyclopentyl]carbonate

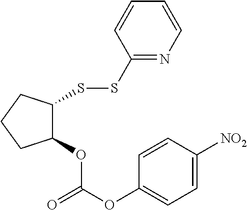 [SMILES: O=C(Oc1ccc([N+](=O)[O-])cc1)O[C@H]1CCC[C@@H]1SSc1ccccn1]

### Step 1. Synthesis of 2-benzylthiocyclopentanol

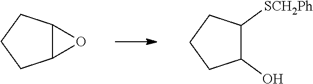

A 5 L reactor was charged with zinc D-tartrate (500 g), dichloromethane (2 L), benzylmercaptan (127 g; 1 mol) and cyclopentene oxide (100 g; 1.1 eq.). The reaction was placed under an Argon atmosphere. The mixture was stirred for 15 days (providing >99% mercaptan conversion) and filtered. The filtrate was evaporated and transferred to a smaller flask using ethyl acetate. Evaporation yielded a turbid oil (210 g; 58% e.e.). The oil was distilled under deep vacuum using a standard Claisen distillation setup. Four fractions were obtained at 102-108° C./0.07-0.15 mbar.

### Step 2. Synthesis of (S,S)-2-benzylthiocyclopentanol

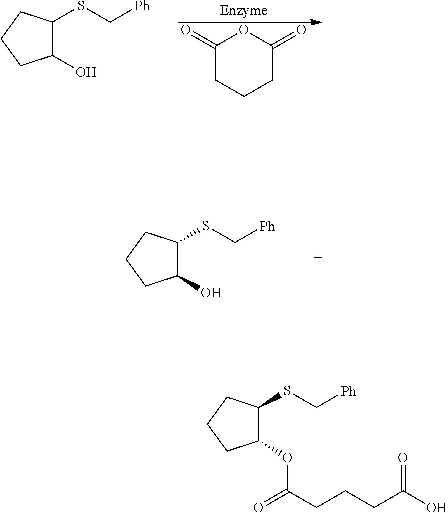 [SMILES: OC1CCCC1SCc1ccccc1 ; O=C1CCCC(=O)O1 ; O[C@H]1CCC[C@@H]1SCc1ccccc1 ; O=C(O)CCCC(=O)O[C@@H]1CCC[C@H]1SCc1ccccc1]

Enriched 2-benzylthiocyclopentanol (88 g; 58 e.e.; 95% pure distillation prerun) was placed in a 1 L flask. MTBE (0.6 L), glutaric anhydride (23 g; 0.2 mol) and immobilized enzyme (CaLB-ADS4; 10 g) were added. The mixture was stirred at 180 rpm using a mechanical stirrer for 1 day. After 18 h the minor (R,R)-enantiomer was completely removed. The reaction mixture was decanted and the enzyme reused in the next procedure. The decanted solution was washed with aqueous ammonia (0.25 L; 2 M) and sodium carbonate (0.1 L; 1.25 M). The organic phase was isolated, dried on sodium sulfate and evaporated to a slightly turbid colorless oil (64 g; 73%). This oil was Kugelrohr-distilled in 2 portions, yielding 54 g of 95% GC purity oil, showing 99.8% e.e.

Alternatively, enriched 2-benzylthiocyclopentanol (104 g; 57% e.e.; 97.6% pure distillation main run) was placed in a 1 L flask. MTBE (0.5 L), glutaric anhydride (23 g; 0.2 mol), and immobilised enzyme (CaLB-ADS4; 10 g recovered and 10 g fresh) were added. The mixture was stirred at 180 rpm using a mechanical stirrer for 1 day. After 24 h the minor (R,R)-enantiomer was completely removed. The reaction mixture was filtered. The filtrate was washed with aqueous ammonia (0.25+0.05 L; 2 M). The organic phase was isolated, dried on sodium sulfate and evaporated to a slightly turbid colourless oil (80 g; 77%). This oil was Kugelrohr-distilled (oven 175° C./0.06 mbar) in 3 portions, yielding 74 g of 97.5% GC purity oil, showing greater than 99.8% e.e. [α]_{D}: +14°° (c=5 in DCM); [α]_{D}: −17° (c=1 in MeOH).

### Step 3. Synthesis of (S,S)-2-Mercaptocyclopentanol

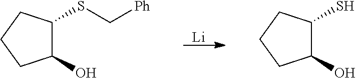

Distilled (S,S)-2-benzylthiocyclopentanol (21 g; 100 mmol) was placed in a dry 250 ml round bottom flask under argon atmosphere and dissolved in 200 mL anhydrous 2-methyltetrahydrofuran. Under mechanical stirring, lithium grains (2.8 g total; 0.4 mol) were added. The reaction was cooled in a water bath. Overnight stirring at ambient temperature yielded a grey slurry. GC of a sample showed 15 area % product. The mixture was quenched by slow addition of MeOH (25 mL) in 2 h. Copious gas evolution was observed. GC sample showed higher conversion after quench. Further quench with aqueous ethanol produced more foaming.

The reaction mixture was diluted in 0.25 L water. The organic phase was removed and the aqueous phase was washed with EtOAc. The washed aqueous phase was acidified with 30 g of solid citric acid and extracted with EtOAc (200+100 mL). The extracts were concentrated under reduced pressure to provide a pungent oil (8 g). Kugelrohr distillation provided a colorless oil (1.39 g+3.95 g).

Alternatively, distilled (S,S)-2-benzylthiocyclopentanol (21 g; 100 mmol) was mixed with MeOH (3.2 g; 100 mmol) and dissolved in anhydrous 2-methyltetrahydrofuran (25 mL). This solution was added dropwise in 2.5 h to a suspension of lithium grains (2.8 g; 0.4 mol) in anhydrous 2-methyltetrahydrofuran (250 mL) in a dry 1 L round bottom flask under argon atmosphere under mechanical stirring. After 4 h a solution of MeOH (3.5 ml) in 2-methyltetrahydrofuran (50 mL) was added dropwise in 1 h. The mixture was quenched by slow addition of MeOH (10 mL) in 2-MeTHF (40 mL) in 1 h. Copious gas evolution was observed.

The reaction mixture was diluted in water (0.3 L). The organic phase was removed and the highly basic aqueous was washed with 2-MeTHF. The washed aqueous phase was acidified with solid citric acid (30 g) and extracted with EtOAc (250+100 ml). The extracts were evaporated under reduced pressure to provide a pungent oil (7.5 g). Kugelrohr distillation [oven 125-135° C./18 mbar] provided a colorless oil (7.0 g). GC 99.5%. [59 mmol; 59%]; [α]_{D}: +49° (c=1 in MeOH).

### Step 4. Synthesis of (S,S)-2-(Pyridin-2-yldisulfaneyl)cyclopentanol

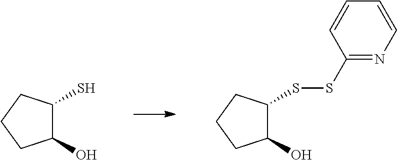 [SMILES: O[C@H]1CCC[C@@H]1S ; O[C@H]1CCC[C@@H]1SSc1ccccn1]

(S,S)-2-Mercaptocyclopentanol (5.9 g; 50 mmol) was dissolved in methanol (50 mL) under Argon and added drop-wise over 1 h 30 m to a solution of dipyridyldisulfide (10.9 g; 49 mmol) in methanol (70 mL). After 5 hours of additional stirring, the reaction mixture was evaporated to dryness and the residue (17 g) mixed with MTBE (250 ml) and sodium hydroxide solution (0.2 L; 1 M). The organic phase was washed with sodium hydroxide (50 mL, 1 M), water (50 mL), sodium bicarbonate solution (25 mL) and brine (25 mL). The organic extract was dried on sodium sulfate and evaporated to a yellow oil (11.1 g; quantitative yield); HPLC: 100% e.e., 8.6 area % DPDS.

Part of the oil (4 g) was mixed with MTBE (8 mL) and pentane until cloudy. This mixture was cooled overnight to −25° C. in freezer; oil precipitation was observed with no enrichment/depletion of impurities according to HPLC.

The oil was recovered and purified by column chromatography on 100 g silica. The column was eluted with 8:2 hexane/ethyl acetate followed by 7:3 hexane/ethyl acetate.

The product was isolated as a slightly turbid oil, with an overall recovery of 80%. GC: >99.5%; HPLC: 99.8%; Chiral HPLC: 100% e.e.; [α]_{D}: −46° (c=1 in EtOH).

### Step 5. Synthesis of 4-Nitrophenyl[(S,S)-2-(pyridin-2-yldisulfaneyl)cyclopentyl]carbonate

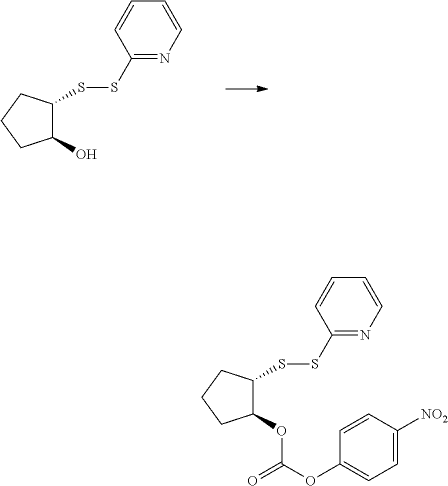 [SMILES: O[C@H]1CCC[C@@H]1SSc1ccccn1 ; O=C(Oc1ccc([N+](=O)[O-])cc1)O[C@H]1CCC[C@@H]1SSc1ccccn1]

Crude (S,S)-2-(pyridin-2-yldisulfaneyl)cyclopentanol (7 g; about 25 mmol) was placed in a dry flask under argon atmosphere and dissolved in anhydrous dichloromethane (80 mL). Pyridine (5 ml; 60 mmol; 3 eq.) was added. A solution of 4-nitrophenyl chloroformate (4.5 g; 23 mmol) in anhydrous dichloromethane (20 mL) was added dropwise under argon over about 1 hour at ambient temperature. The mixture was quenched with water, washed with dilute hydrochloric acid (100+50 mL; 1 M), water, and sodium bicarbonate solution. Filtration over a plug of sodium sulfate and evaporation under reduced pressure provided the title compound as a turbid oil (10 g, quantitative yield). HPLC: 85% purity.

Conjugates comprising the cyclopentyl linker moiety can be prepared according to the processes disclosed in Example 11 of U.S. patent application Ser. No. 16/925,094 as well as Example 9 herein.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

What is claimed is:

1. A process for preparing a compound of Formula (A1) 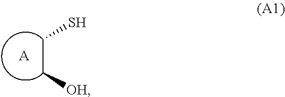
or a salt thereof, wherein ring A is C₅₋₇ cycloalkyl or 5-7 membered heterocycloalkyl, comprising:
a) treating a compound of Formula (A4) 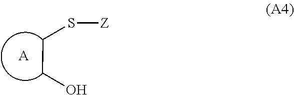
or a salt thereof, wherein Z is a protecting group, with Ak1, wherein Ak1 is an acylating reagent, in the presence of an enzyme to provide a mixture of a compound of Formula (A2) and a compound of Formula (A3); 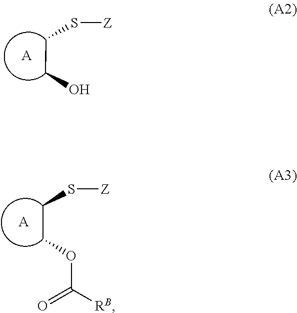
or salts thereof; wherein R^{B} is C₁₋₆ alkyl optionally substituted with COOH; and
b) deprotecting the compound of Formula (A2), or a salt thereof, to provide a compound of Formula (A1), or a salt thereof.

2. The process of claim 1, wherein Ring A is a C₅₋₇ cycloalkyl.

3. The process of claim 1, wherein Ring A is cyclohexyl.

4. The process of any one of claims 1-3, wherein the Compound of Formula (A1) is Compound 1:

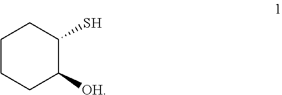

5. The process of any one of claims 1-4, wherein Z is —CH₂R^{A}, wherein R^{A} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents independently selected from C₁₋₄ alkyl, halo, CN, NO₂, OH, and OCH₃.

6. The process of claim 5, wherein R^{A} is phenyl.

7. The process of any one of claims 1-6, wherein Ak1 is glutaric anhydride, succinic anhydride, or isopropenyl acetate.

8. The process of any one of claims 1-6, wherein Ak1 is glutaric anhydride.

9. The process of any one of claims 1-8, wherein R^{B} is CH₃, CH₂CH₂COOH, or CH₂CH₂CH₂COOH.

10. The process of any one of claims 1-8, wherein R^{B} is CH₂CH₂CH₂COOH.

11. The process of any one of claims 1-10, wherein the enzyme is a lipase enzyme derived from Candida antarctica.

12. The process of any one of claims 1-10, wherein the enzyme is lipase B derived from Candida antarctica.

13. The process of any one of claims 1-12, wherein the enzyme is immobilized on a solid support.

14. The process of claim 13, wherein the solid support comprises acrylic beads.

15. The process of any one of claims 1-14, wherein the treating of a compound of Formula (A4) with Ak1 is performed at a temperature between about 15° C. and about 20° C.

16. The process of any one of claims 1-14, wherein the treating of a compound of Formula (A4) with Ak1 is performed at room temperature.

17. The process of any one of claims 1-16, wherein the treating of a compound of Formula (A4) with Ak1 is performed for a period of about 6 h to about 24 h.

18. The process of any one of claims 1-16, wherein the treating of a compound of Formula (A4) with Ak1 is performed for a period of about 16 h.

19. The process of any one of claims 1-18, wherein the treating of a compound of Formula (A4) with Ak1 is performed in the presence of S1, wherein S1 is a solvent.

20. The process of claim 19, wherein S1 is an ether solvent.

21. The process of claim 19, wherein S1 is methyl tert-butyl ether.

22. The process of claim 19, wherein S1 is 2-methyltetrahydrofuran.

23. The process of any one of claims 1-22, further comprising the step of separating the compound of Formula (A2) from the compound of Formula (A3).

24. The process of claim 23, wherein the separating comprises treating the mixture with an aqueous base and separating the aqueous layer from the mixture.

25. The process of claim 24, wherein the aqueous base is aqueous sodium carbonate.

26. The process of any one of claims 1-25, wherein Z is —CH₂R^{A}, and the deprotecting comprises reducing the compound of Formula (A2) with RA1, wherein RA1 is a reducing agent.

27. The process of claim 26, wherein RA1 is lithium metal, sodium metal, or calcium metal.

28. The process of claim 26, wherein RA1 is lithium metal.

29. The process of any one of claims 26-28, wherein the reducing is carried out in the presence of S2, wherein S2 is a solvent.

30. The process of claim 29, wherein S2 is an ether solvent.

31. The process of claim 29, wherein S2 is 2-methyltetrahydrofuran.

32. The process of any one of claims 1-31, wherein Compound 1 is isolated in greater than 75% enantiomeric excess.

33. The process of any one of claims 1-31, wherein Compound 1 is isolated in greater than 90% enantiomeric excess.

34. The process of any one of claims 1-31, wherein Compound 1 is isolated in greater than 95% enantiomeric excess.

35. The process of any one of claims 1-34, wherein the compound of Formula (A4) is prepared by a process comprising reacting a compound of Formula (A5) 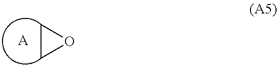
or a salt thereof, with R^{A}CH₂SH (Formula (6)), or a salt thereof, to provide the compound of Formula (A4) or a salt thereof.

36. The process of claim 35, wherein the reacting of the compound of Formula (A5) or a salt thereof with R^{A}CH₂SH (Formula (6)), or a salt thereof, is performed in the presence of M1, wherein M1 is a metal catalyst.

37. The process of claim 36, wherein M1 is a zinc salt.

38. The process of claim 36, wherein M1 is zinc (D)-tartrate.

39. The process of any one of claims 35-38, wherein the reacting of the compound of Formula (A5) or a salt thereof with R^{A}CH₂SH (Formula (6)) is performed in the presence of B1, wherein B1 is a base.

40. The process of claim 39, wherein B1 is an alkoxide base.

41. The process of claim 39, wherein B1 is sodium ethoxide.

42. The process of any one of claims 36-41, wherein the reacting of the compound of Formula (A5) with the compound of Formula (6) is performed in the presence of S3, wherein S3 is a solvent.

43. The process of claim 42, wherein S3 is a halogenated solvent or an ether solvent.

44. The process of claim 42, wherein S3 is dichloromethane.

45. The process of claim 42, wherein S3 is 2-methyltetrahydrofuran.

46. The process of any one of claims 35-45, wherein the compound of Formula (A4) is isolated in greater than 25% enantiomeric excess.

47. The process of any one of claims 35-45, wherein the compound of Formula (A4) is isolated in greater than 50% enantiomeric excess.

48. The process of any one of claims 35-45, wherein the compound of Formula (A4) is isolated in greater than 70% enantiomeric excess.

49. A process for preparing a compound of Formula (1) 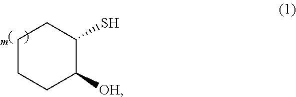
or a salt thereof, wherein m is 0, 1, or 2, comprising:
a) treating a compound of Formula (4) 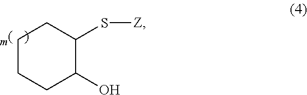
or a salt thereof, wherein Z is a protecting group, with Ak1, wherein Ak1 is an acylating reagent, in the presence of an enzyme to provide a mixture of a compound of Formula (2) and a compound of Formula (3); 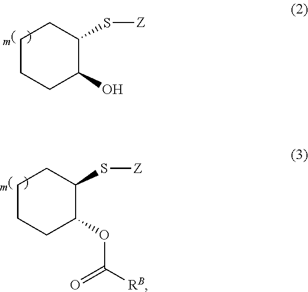
or salts thereof; wherein R^{B} is C₁₋₆ alkyl optionally substituted with COOH; and
b) deprotecting the compound of Formula (2), or a salt thereof, to provide a compound of Formula (1), or a salt thereof.

50. The process of claim 49, wherein m is 1.

51. A process for preparing a compound of Formula (8): 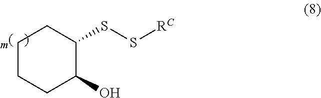
or a salt thereof, wherein m is 0, 1, or 2, and R^{C} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents independently selected from C₁₋₄ alkyl, halo, CN, NO₂, OH, and OCH₃, comprising reacting a compound of Formula (7) 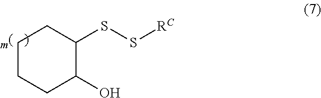
or a salt thereof, with Ak2, wherein Ak2 is an acylating reagent, in the presence of an enzyme to provide a mixture of a compound of Formula (8) and a compound of Formula (9); 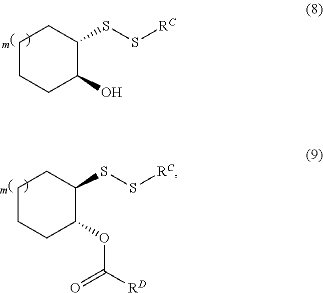
or salts thereof, wherein R^{D} is C₁₋₆ alkyl optionally substituted with COOH.

52. A process for preparing a compound of Formula (A-I): 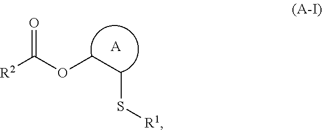
or a pharmaceutically acceptable salt thereof, wherein
ring A is C₅₋₇ cycloalkyl or 5-7 membered heterocycloalkyl;
R¹ is a targeting moiety; and
R² is a therapeutic moiety;
comprising:
a) reacting a compound of Formula (A1), or a salt thereof, prepared by the process of any one of claims 1-48, with R^{C}—S—S—R^{C} to provide a compound of Formula (A8) 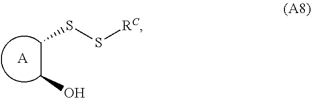
or a salt thereof, wherein R^{C} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents independently selected from C₁₋₄ alkyl, halo, CN, NO₂, OH, and OCH₃;
b) reacting a compound of Formula (A8), or a salt thereof, with R^{E}OC(O)R^{F}, to provide a compound of Formula (A-1B) 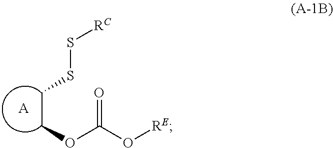
or a salt thereof, wherein:
R^{E} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃; and
R^{F} is halo or OR^{F1}, wherein OR^{F1} is C₆₋₁₀ aryl or 5-10 membered heteroaryl; wherein the 5-10 membered heteroaryl has at least one ring-forming carbon atom and 1, 2, 3, or 4 ring-forming heteroatoms independently selected from N, O, and S; and wherein the C₆₋₁₀ aryl and 5-10 membered heteroaryl are each optionally substituted with 1, 2, 3, 4, or 5 substituents selected from C₁₋₄ alkyl, halo, CN, NO₂, OH, and OCH₃;
c) reacting the compound of formula (A-1B) or a salt thereof, with R²H to provide a compound of Formula (A-1C) 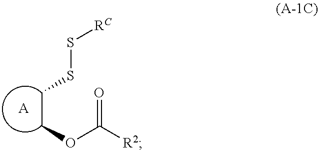
or a salt thereof; and
d) reacting a compound of Formula (A-1C), or a salt thereof, with R¹H to provide a compound of Formula (A-I).

53. The process of claim 52, wherein the compound of Formula (A-I) has Formula (A-I)′: 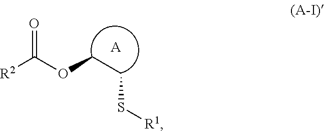
or a pharmaceutically acceptable salt thereof.

54. The process of claim 52 or 53, wherein R¹ is a peptide comprising at least one of the following sequences:
(SEQ ID NO. 1; Pv1)
ADDQNPWRAYLDLLFPTDTLLLDLLWCG,
(SEQ ID NO. 2; Pv2)
AEQNPIYWARYADWLFTTPLLLLDLALLVDADECG,
and
(SEQ ID NO. 3; Pv3)
ADDQNPWRAYLDLLFPTDTLLLDLLWDADECG,
and wherein R¹ is attached to the S atom of the compound of Formula (I) through a cysteine residue of R¹.

55. The process of claim 52 or 53, wherein R¹ is ADDQNPWRAYLDLLFPTDTLLLDLLWCG (SEQ ID NO: 1; Pv1), and wherein R¹ is attached to the S atom of the compound of Formula (I) through a cysteine residue of R¹.

56. The process of any one of claims 52-55, wherein R² is a topoisomerase I targeting moiety.

57. The process of any one of claims 52-55, wherein R² is:

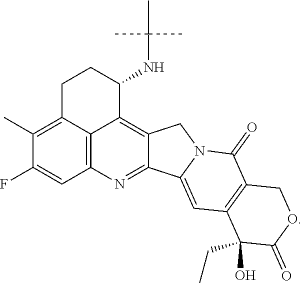

58. The process of claim 52, wherein the compound of Formula (A-I) is

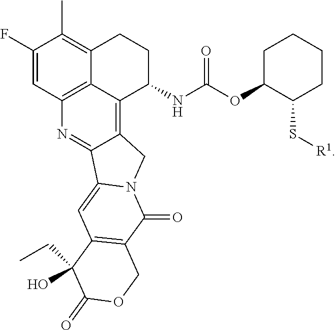

59. A compound of Formula (A1), or a salt thereof, prepared by the process of any one of claims 1-48.

60. A compound of Formula (1), or a salt thereof, prepared by the process of claim 49 or 50.

61. A compound of Formula (8), or a salt thereof, prepared by the process of claim 51.

62. A compound of Formula (I), or a salt thereof, prepared by the process of any one of claims 52-58.


SEQUENCE LISTING (5 sequences)

SEQ ID NO: 1 - protein, 28 aa, Artificial
  synthetic peptide
ADDQNPWRAYLDLLFPTDTLLLDLLWCG

SEQ ID NO: 2 - protein, 35 aa, Artificial
  synthetic peptide
AEQNPIYWARYADWLFTTPLLLLDLALLVDADECG

SEQ ID NO: 3 - protein, 32 aa, Artificial
  synthetic peptide
ADDQNPWRAYLDLLFPTDTLLLDLLWDADECG

SEQ ID NO: 4 - protein, 39 aa, Artificial
  synthetic peptide
AAEQNPIYWARYADWLFTTPLLLLDLALLVDADEGTKCG

SEQ ID NO: 5 - protein, 37 aa, Artificial
  synthetic peptide
AAEQNPIYWARYADWLFTTPLLLLDLALLVDADEGTC